(19) United States
(12) Patent Application Publication
(10) Pub. No.: US 2023/0346580 A1
(43) Pub. Date: Nov. 2, 2023

(54) DILATORS AND SHEATHS WITH EXPANSION FEATURES

(71) Applicant: EDWARDS LIFESCIENCES CORPORATION, Irvine, CA (US)

(72) Inventor: Maxwell Harrison Fine, Costa Mesa, CA (US)

(21) Appl. No.: 18/220,730

(22) Filed: Jul. 11, 2023

Related U.S. Application Data
(63) Continuation of application No. PCT/US2022/012684, filed on Jan. 17, 2022.
(60) Provisional application No. 63/246,674, filed on Sep. 21, 2021.
(60) Provisional application No. 63/138,736, filed on Jan. 18, 2021.

(51) Int. Cl. A61F 2/962

(52) U.S. Cl. CPC A61F 2/962

(57) ABSTRACT

Disclosed herein are introducer systems including dilators, sheaths, methods, and tools for making dilators and sheaths. The introducer systems disclosed herein include features to assist in expansion of a sheath, particularly at proximal or distal regions of the sheath. Methods of making the dilators and sheaths include steps to allow or cause expansion of certain regions of the sheath during fabrication, and/or steps to allow or cause expansion of certain regions of the sheath during delivery of a medical device.

### RELATED APPLICATIONS

This application is a continuation of International Application No. PCT/US2022/012684, filed Jan. 17, 2022, which claims the benefit of U.S. Provisional Applications 63/138,736, filed Jan. 18, 2021, and 63/246,674, filed Sep. 21, 2021. Each of the aforementioned applications is incorporated by reference in its entirety for all purposes.

### FIELD

This disclosure relates to medical device delivery systems, and more particularly to dilators and sheaths for transvascular procedures.

### BACKGROUND

Introducer systems are the part of a medical device delivery system used to access the vascular system during a transvascular medical device delivery procedure (i.e., to assist in introducing the medical device). To use an introducer system, a tapered dilator is placed inside a sheath, and the two are then inserted into the vasculature over a guide wire at an access site. The dilator helps to facilitate the insertion of the sheath into the arteriotomy by gradually tapering at the distal end from an outer diameter slightly larger than the guidewire, to an outer diameter just smaller than the distal tip of the sheath. The transition from the dilator shaft to the tip of the expandable sheath should be minimized in order to prevent trauma to the tissue as the system is inserted. If the dilator shaft is sufficiently smaller in outer diameter than the sheath tip, the sheath may catch on the vessel, causing shear forces and potential damage. To make the transition from dilator to sheath as atraumatic as possible, the sheath tip should fit tightly around the dilator. The transition must be smooth with no raised lips or edges.

After the sheath is fully inserted into the body, the dilator is removed so that medical devices can be passed through the lumen of the sheath. A sheath inserted via the femoral artery is designed for the tip to rest in the abdominal aorta. Here, the vessel diameter is much larger than the outer diameter of the sheath such that the sheath tip does not interact with the walls of the vessel. At this point in the procedure, the sheath tip is less of a risk to the vasculature, and the atraumatic design considerations are not as important. Instead, the role of the sheath is now to facilitate insertion and removal of interventional devices that track further into the vessel or into the heart. In order to do so, the tip must easily expand to accommodate devices of a larger outer diameter to exit the sheath. If the expansion of the tip is too difficult, the clinician may have issues advancing the device fully through the sheath, compromising the procedure. Similarly, if the sheath cannot expand sufficiently to accommodate a device during retrieval, or if the sheath tip does not have enough column strength to allow device retrieval without buckling, the clinician may have difficulty removing the device, potentially leading to surgery.

Finally, after all interventional devices are retrieved from the sheath, the sheath must be removed from the body. The tip must be free of any protrusions or sharp edges to be pulled back through the smaller portions of the vessel once again, without causing damage. Overall, the sheath tip must be low profile and smooth during sheath insertion, expand easily during insertion and retrieval of interventional devices, then again be atraumatic during sheath removal. These contradicting requirements currently cause challenges such as: high push force required to move the delivery system through the sheath tip, tip tears or separation during expansion, radially protruding sharp edges of tip material after tip expansion, high balloon retrieval forces, difficulty retrieving burst balloons, difficulty retrieving crimped valve (bailout), step or protrusion in the tip after expansion, leading to difficulty removing the sheath.

A smooth transition between the sheath and the rest of the system is also desired at the proximal end. A sheath can be used to promote the smooth transition from the sheath to a sheath hub while also providing a seal between the sheath and the sheath hub. During operation of the sheath and dilator, it is possible that the sheath can become loose or detach from the rest of the system due to an uneven diameter at the proximal end of the sheath. Looseness or detachment of the sheath can interrupt the smooth transition from the sheath to the sheath hub and disrupt the seal. This disruption can cause the sheath and dilator to become misaligned, prolonging procedure time and recovery and increasing risk of trauma to vessels and heart tissue. During manufacturing, the proximal end of the sheath must be enlarged and securely and reliably coupled to the sheath hub. Improved manufacturing methods are desired to reduce assembly and processing time while improving engagement between the sheath and the sheath hub.

Accordingly, there remains a need for further improvements in introducer systems used to implant valves and other prosthetic devices and their corresponding manufacturing processes.

### SUMMARY

This sheath and dilator system disclosed herein removes the need for the crimped valve to expand the sheath tip during interventional device insertion, and the delivery system balloon (or bailout valve) to expand the sheath tip during retrieval. These device features are designed for their respective interventional procedures, and not to interface in this manner with the sheath. Instead, the sheath tip expansion can be performed by an expansion feature on the dilator, which would be specifically designed for this purpose. The dilator features would then expand the sheath tip safely after being placed in the abdominal aorta. The safe expansion of the sheath tip would eliminate traumatic features to the tip and allow for easier passage of the delivery system through the sheath, in both insertion and retrieval. This enables the sheath tip to be optimized for the post-insertion phase, supporting lower push force through the tip, no tip damage, easy balloon retrieval, improved ability to retrieve burst balloons, improved ability to perform bailout procedures, and minimized vessel trauma upon sheath retrieval.

The dilators disclosed herein include a shaft with a tapered distal region that narrows distally towards a distal tip. An expander is positioned on the shaft adjacent to the distal tip. In some examples, the expander is positioned proximal to the tapered distal region. The expander can take a compressed configuration, with the expander compressed against the shaft. The expander can also take an expanded configuration, where it extends radially away from the shaft. In some aspects, in the expanded configuration, the expander extends away from the shaft in multiple radial directions. In some examples, the expander extends away from the shaft in all radial directions. In some examples, a proximal region of the dilator can include a stopping feature positioned a fixed distance from the expander.

In some embodiments, the expander is integrally formed with the shaft. In others, the expander is coupled to the shaft. For example, the expander can include at least one tubular portion that extends circumferentially around the shaft. The tubular portion of the expander can be bonded to the shaft.

In some embodiments, the expander can include a shape memory material. The expander can take the expanded configuration upon a change in the surroundings due to the shape memory material. For example, the expander can include a plurality of longitudinally extending slits. The longitudinally extending slits can extend proximally from a distal end of the expander. The plurality of longitudinally extending slits can divide the expander into longitudinally extending arms that move away from the shaft upon a change in the surroundings due to the shape memory material. In some embodiments, the expander can include one or more folds in the compressed configuration that unfold upon a change in the surroundings. For example, the unfolding of the folds can form a funnel in the expanded configuration.

In some embodiments, the expander can include two or more extensions that point toward the proximal region of the dilator. The expander with two or more extensions pointing toward the proximal region of the dilator can be biased toward the expanded configuration, with the extensions extending radially away from the shaft, such that external pressure must be applied to the two or more extensions to keep the expander in the compressed configuration. Each of the two or more extensions can include a hinge that couples the extension to the expander, and the extensions can be rotatable around the hinge to point toward the distal region of the dilator.

The dilator can also include a proximal region with a stopping feature. The stopping feature is positioned a fixed distance from the expander. In some embodiments, the stopping feature has a larger diameter than the shaft. Alternatively or additionally, the stopping feature can be a visual indicator.

The medical device delivery systems disclosed herein can include a sheath with a distal portion that is configured to expand radially outwardly. For example, the sheath can include two or more weakened, longitudinally extending areas that extend proximally from the distal end of the sheath. When used with a dilator having extensions that point toward the proximal region of the dilator, the extensions can be positioned just distal to the distal end of the sheath. The extensions of the dilator's expander can be aligned to the weakened longitudinal areas of the distal end of the sheath using alignment indicators. The proximal portion of the sheath can include a first alignment indicator, and the proximal region of the dilator can include a second alignment indicator. Alignment of the first alignment indicator with the second alignment indicator corresponds to alignment of the weakened areas of the distal portion of the sheath with the extensions of the expander. Proximal retraction of the dilator causes then extensions to apply force on the weakened areas of the sheath, tearing them as it is retracted.

Methods of making the dilators disclosed herein include forming a shaft with a tapered distal region, coupling an expander to the shaft adjacent to a distal tip of the shaft, and adding a stopping feature to a proximal region of the shaft. In some examples, the expander is coupled proximal to the tapered distal region of the shaft. In some examples, a tubular portion of the expander is coupled such that it extends circumferentially around the shaft.

The methods of making the dilators disclosed herein can also include forming an expander. The expander can be formed, for example, of a shape memory material. Some method examples include cutting longitudinally extending slits such that they extend proximally from a distal end of the expander. Some method examples include forming a funnel with a shape memory material on a distal side of the expander. The funnel can be folded into a tubular shape. In some embodiments, the expander can be formed to include two or more extensions biased to point in a proximal direction and biased to extend radially away from the shaft, such that they are rotatable about a hinge to point in a distal direction.

Some methods of making the dilators disclosed herein can include forming the stopping feature at the proximal region of the shaft to have a larger diameter than the shaft itself. In some methods, the stopping feature is a stopping point that is marked directly on the shaft as a visual indicator.

Methods of delivering medical devices are also disclosed herein. The methods can include inserting a tapered dilator with an expander into a sheath, then inserting the tapered dilator and the sheath into the vasculature of the subject. The methods further include expanding or tearing a distal portion of the sheath with the expander and removing the dilator from the sheath. The medical device is then delivered to the subject through the sheath.

Inserting the tapered dilator into the sheath can include axially translating the dilator until a stopping feature on the dilator is a predetermined distance from a distal end of the sheath. In some examples, inserting the tapered dilator into the sheath comprises axially translating the dilator until the stopping feature abuts a proximal end of the sheath.

In some methods, the tapered dilator is axially translated through the sheath until at least part of the expander is beneath the distal portion of the sheath. The expander can be induced to take an expanded configuration beneath the distal portion of the sheath, thereby expanding a distal portion of the sheath. The expander can take the expanded configuration by, for example, moving longitudinally extending arms of the expander radially away from a shaft of the dilator, or by unfolding one or more folds of the expander.

In some examples of the method of delivering a medical device, the tapered dilator is axially translated through the sheath until at least part of the expander is distal to the distal portion of the sheath. The distal portion of the sheath can be torn by the expander as the dilator is removed from the sheath. The expander can be aligned with the distal portion of the sheath to target the tearing to weakened areas of the distal portion of the sheath. For example, if the expander includes two or more proximally pointing extensions positioned distal to the distal portion of the sheath and aligned to weakened areas of the distal portion of the sheath, as the dilator is removed, the extensions will tear the weakened areas of the sheath and then rotate as they contact the non-weakened portions of the sheath. The expander is then compressed beneath the sheath, with the extensions pointing distally, as the dilator is further removed from the subject through the lumen of the sheath.

Methods of making a sheath are disclosed herein. The methods can include forming an inner sleeve having a proximal end, a distal end, and an inner sleeve body that extends between the proximal end and the distal end defining a central opening and forming an outer sleeve having a proximal end, a tapered distal end and an outer sleeve body that extends between the proximal end and the distal end defining a central opening. The methods can further include forming a plurality of longitudinal slits in the inner sleeve, the slits extending from the proximal end of the inner sleeve along a portion of the inner sleeve body. A plurality of longitudinal slits can be formed in the outer sleeve, the slits extending from the proximal end of the outer sleeve along a portion of the outer sleeve body. A plurality of window cuts can be formed in the outer sleeve, the window cuts extending from the distal end of the outer sleeve along a portion of the outer sleeve body. The proximal end of the inner sleeve is disposed over a flaring tool and a portion of the inner sleeve is disposed in the central opening of the outer sleeve. The outer sleeve and the inner sleeve are then coupled together forming a coupled proximal end. Coupling the outer sleeve and the inner sleeve together forms a uniform thickness about the circumference of the coupled proximal end.

In some examples of the methods of making a sheath, forming a plurality of longitudinal slits in the inner sleeve comprises placing the proximal end of the inner sleeve over a split cut guide and sliding a cutting tool along the slit cut guide. Each of the plurality of longitudinal slits of the inner sleeve can extend along a line generally parallel with a longitudinal axis of the inner sleeve. Forming a plurality of longitudinal slits in the outer sleeve can include placing the proximal end of the outer sleeve over a slit cut guide and sliding a cutting tool along the slit cut guide. Each of the plurality of longitudinal slits of the outer sleeve can extend along a line generally parallel with a longitudinal axis of the outer sleeve. Forming a plurality of window cuts in the outer sleeve can include placing the distal end of the outer sleeve over a window cut guide and sliding a cutting tool along the window cut guide. In some examples of the methods, the length of each of the slits is greater than the length of each window cut, such that placing the inner sleeve and the outer sleeve over the flaring tool causes the slits to expand and create the tapered shape of the coupled proximal end.

Some example methods of making a sheath include longitudinally aligning the proximal end of the inner sleeve and the proximal end of the outer sleeve. The outer sleeve can be positioned such that the slits of the inner sleeve are circumferentially spaced from the slits of the outer sleeve. In some example methods, the outer sleeve is coupled to the inner sleeve by tacking. In some example methods, the outer sleeve is coupled to the inner sleeve by heat processing. Some examples include disposing a heat shrink sleeve (for example, a PTFE heat shrink) around the outer sleeve and a portion of the inner sleeve that includes the proximal end of the inner sleeve. The outer sleeve can be reflowed with the inner sleeve. In some examples, the outer sleeve and the inner sleeve are reflowed around a flaring tool, forming a flared surface at the coupled proximal end. The heat shrink sleeve, if used, can then be removed. Some example methods can include forming a fillet surface transition at the coupled proximal end. The coupled proximal end can be disposed between a sheath hub and a sheath hub cap to secure the proximal end to the sheath hub.

The sheaths disclosed herein include an inner sleeve having a flared proximal end, a distal end and an inner sleeve body that extends between the proximal end and the distal end and defines a central opening. The inner sleeve includes a plurality of circumferentially spaced slits extending from the proximal end along a portion of the inner sleeve body. The sheaths further include an outer sleeve having a proximal end, a tapered distal end and an outer sleeve body that extends between the proximal end and the distal end and defines a central opening. The outer sleeve includes a plurality of circumferentially spaced slits extending from the proximal end along a portion of the outer sleeve body. The outer sleeve also includes a plurality of window cuts that extend from the distal end along a portion of the outer sleeve body. A portion of the proximal end of the inner sleeve is disposed in the central opening of the outer sleeve, such that the proximal end of the inner sleeve and the proximal end of the outer sleeve are longitudinally aligned. The inner sleeve and the outer sleeve are coupled together forming a coupled proximal end.

In some examples, the window cuts are circumferentially spaced apart from the slits around the outer sleeve. A window cut can be disposed on either side of each slit. Each slit can be centered between an adjacent pair of window cuts. In some examples, the length of each slit is greater than a length of each window cut. In some examples, each slit extends along the outer sleeve body beyond a proximal end of each cut. In some examples of the sheaths disclosed herein, the outer sleeve can include a recess along a length of outer sleeve that extends circumferentially from either side of each of the circumferentially spaced slits.

In some examples of the sheaths disclosed herein, each of the plurality of longitudinal slits of the inner sleeve extend along a line generally parallel with a longitudinal axis of the inner sleeve. In some examples, each of the plurality of longitudinal slits of the inner sleeve has a same length along the inner sleeve. In some examples, each of the plurality of longitudinal slits of the outer sleeve extend along a line generally parallel with a longitudinal axis of the inner sleeve. In some examples, each of the plurality of longitudinal slits of the outer sleeve has a same length along the outer sleeve. In some examples of the sheaths disclosed herein, the slits of the inner sleeve are circumferentially spaced apart from the slits in the outer sleeve.

The number of slits in the inner sleeve can be equal to the number of slits in the outer sleeve. In some examples, the number of slits in the outer sleeve is equal to the number of window cuts in the outer sleeve. In some examples, there are twice as many longitudinal slits as window cuts on the outer sleeve. In various examples, the inner sleeve can include 3 slits or 4 slits. In various examples, the outer sleeve can include 3 slits or 4 slits. In various examples, the outer sleeve can include 3 window cuts or 4 window cuts.

In some examples, the inner sleeve and the outer sleeve each have a uniform thickness. For example, the uniform thickness can be double the thickness of the inner sleeve. The inner sleeve and outer sleeve can be formed from the same material.

In some examples of the sheaths disclosed herein, the coupled proximal end comprises a flared diameter. The flared diameter further can include a fillet transition surface. Some examples include a sheath hub and a sheath hub cap coupled to the coupled proximal end.

A cutting guide used for forming the sheaths disclosed herein can include: a slit cut guide having a first cylindrical body and a plurality of longitudinal grooves circumferentially disposed about the first cylindrical body, a window cut guide having a second cylindrical body and plurality of V-shaped grooves circumferentially disposed about the second cylindrical body, and a central body. The slit cut guide and the window cut guide can each be coupled to the central body. Each of the plurality of longitudinal grooves can define a guide channel shaped to guide a cutting tool to form a longitudinal slit in a tube disposed about the slit cut guide. Each of the plurality of V-shaped grooves can define guide edges to guide a cutting tool to form a window cut in a tube disposed about the window cut guide.

### DESCRIPTION OF DRAWINGS

The device and methods are explained in even greater detail in the following drawings. The drawings are merely exemplary and certain features may be used singularly or in combination with other features. The drawings are not necessarily drawn to scale.

FIG. 1 is an example dilator comprising an expander.

FIG. 2A is an example dilator inserted into a sheath, with the expander positioned beneath the distal portion of the sheath.

FIG. 2B is the dilator and sheath of FIG. 2A, with the expander in the expanded configuration beneath the distal portion of the sheath.

FIG. 3A is an example of an expander in a compressed configuration.

FIG. 3B is the expander example of FIG. 3A in an expanded configuration.

FIG. 4A is another example of an expander in a compressed configuration.

FIG. 4B is the expander example of FIG. 4A in an expanded configuration.

FIG. 5A is a dilator with an expander as it is being inserted into a sheath.

FIG. 5B is the dilator with the example expander of FIG. 5A fully inserted into the sheath, with extensions from the expander positioned distal to the distal end of the sheath.

FIG. 5C is the dilator with the example expander of FIGS. 5A and 5B as the dilator is proximally withdrawn from the sheath.

FIG. 6 is the distal portion of the sheath with tears left by the extensions of the expander of FIGS. 5A-5C.

FIG. 7 is an example sheath with a representative delivery system.

FIG. 8 is an example of part of a representative delivery system including a sheath, an outer sleeve, a sheath hub, and a sheath hub cap.

FIG. 9 is a partial side view of a sheath coupled to a sheath hub of FIG. 8.

FIG. 10 is a partial side cross section view of the sheath coupled between the sheath hub cap and the sheath hub of FIG. 8.

FIG. 11 is a partial side detail view of the sheath of FIG. 8.

FIG. 12 is a partial side view of the inner sleeve of the sheath of FIG. 8 in a flared configuration.

FIG. 13 is a perspective view of the outer sleeve of the sheath of FIG. 8 in a flared configuration.

FIG. 14 is a distal end view of the outer sleeve of FIG. 15.

FIG. 15 is a side view of the outer sleeve of FIG. 13 rotated such that a slit is centered along the longitudinal axis.

FIG. 16 is a proximal end view of the outer sleeve of FIG. 15.

FIG. 17 is a proximal end view of the outer sleeve of FIG. 18.

FIG. 18 is a side view of the outer sleeve of FIG. 13 rotated such that the portion of the outer sleeve between adjacent slits is centered along the longitudinal axis.

FIG. 19 a distal end view of the outer sleeve of FIG. 18.

FIG. 20 is a perspective view of the outer sleeve of FIG. 13 in an unflared configuration.

FIG. 21 is a side view of the unflared outer sleeve of FIG. 20.

FIG. 22 is a distal end view of the unflared outer sleeve of FIG. 20.

FIG. 23 is a cross section view of the unflared outer sleeve of FIG. 20 taken along section lines A-A in FIG. 24.

FIG. 24 is a distal end view of the un-flared outer sleeve of FIG. 23.

FIG. 25 is a perspective view of a cutting guide device.

FIG. 26 is the distal end view of the cutting device of FIG. 25.

FIG. 27 is a side view of the cutting device of FIG. 25.

FIG. 28 is a proximal end view of the cutting device of FIG. 25.

FIG. 29 is a perspective view of a flaring tool.

FIG. 30 is a distal end view of the flaring tool of FIG. 29.

FIG. 31 is a side view of the flaring tool of FIG. 29.

FIG. 32 is a proximal end view of the flaring tool of FIG. 29.

FIG. 33 is a perspective view of the inner sleeve of FIG. 12 in an uncut an unflared configuration.

FIG. 34 is a perspective view of the outer sleeve of FIG. 13 in an uncut and an unflared configuration.

FIG. 35 is a side view of the outer sleeve FIG. 34.

FIG. 36 is a distal end of view of the outer sleeve of FIG. 34

FIG. 37 is a side view of the combined inner and outer sleeve placed over a flaring tool.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular example or implementation are not limited to that example or implementation, and may be applied to any example or implementation disclosed. It will understood that various changes and additional variations may be made and equivalents may be substituted for elements thereof without departing from the scope of the disclosure or the inventive concept thereof. Certain aspects and features of any given example may be translated to other examples described herein. In addition, many modifications may be made to adapt a particular situation or device to the teachings of the disclosure without departing from the essential scope thereof. Therefore, it is intended that the disclosure not be limited to the particular implementations disclosed herein, but that the disclosure will include all implementations falling within the scope of the appended claims.

Features, integers, characteristics, compounds, chemical moieties, or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

The terms “proximal” and “distal” as used herein refer to regions of the delivery system. A proximal-most point is the point closest to the practitioner during a procedure, while a distal-most point is farthest from the practitioner during a procedure.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

In the field of transvascular procedures, the terms “introducer” and “introducer system” have been used to refer to a dilator alone, a sheath alone, or a system that includes both a dilator and a sheath (and possibly other components). For clarity's sake, the specific terms “dilator” or “sheath” are used herein when discussing components of an introducer system.

As noted above, the sheath and dilator systems disclosed herein eliminate the need for the medical device to expand the sheath tip during interventional device insertion and the delivery system balloon (or bailout valve) to expand the sheath tip during retrieval. Instead, the sheath tip expansion can be performed by an expansion feature on the dilator, which would be specifically designed for this purpose. The developments disclosed herein can be utilized with a variety of different types of expandable sheaths, including, but not limited to, those disclosed in U.S. Pat. Nos. 8,690,936, 8,790,387, 10,639,152, 10,792,471, 10,799,685, 10,327,896, 8,568,472, and International Application No. PCT/US2020/054594, each of which is incorporated herein by reference.

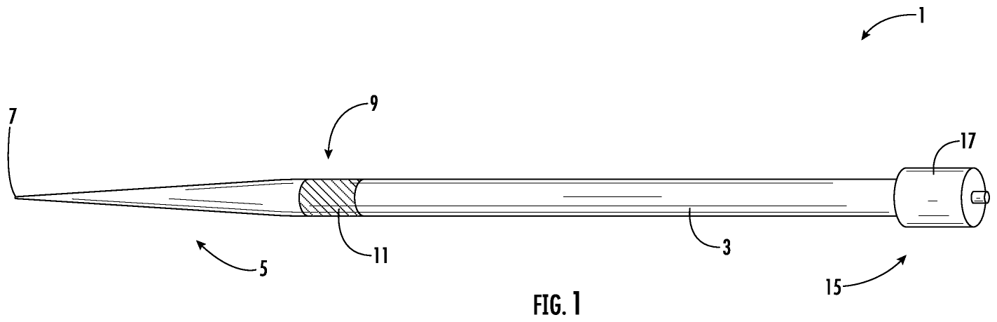

As shown in FIG. 1, the example dilator 1 disclosed herein includes a shaft 3 having a tapered distal region 5, which diminishes in diameter, narrowing distally toward distal tip 7. The dilator 1 includes an expander 9 on shaft 3 positioned adjacent to the distal tip 7. In some examples, the expander 9 is positioned along the dilator 1 near the distal edge of the shaft 3, just proximal to the point at which the shaft 3 begins to taper. In some examples, the expander 9 includes at least a portion 11 that is tubular in shape and extends circumferentially around the shaft 3 of dilator 1. The expander 9 can be coupled to the shaft 3 by conventional techniques such as gluing, bonding or welding, or the expander 9 can be integrally formed with shaft 3. In some examples, tubular portion 11 of the expander is coupled to shaft 3 of the dilator 1. The walls of expander 9 (as defined in a radial direction from an inner surface to an outer surface of expander 9) are relatively thin. For example, the wall thickness of expander 9 can be from about 0.005 inches to about 0.020 inches (including about 0.005 inches, about 0.006 inches, about 0.007 inches, about 0.008 inches, about 0.009 inches, about 0.010 inches, about 0.011 inches, about 0.012 inches, about 0.013 inches, about 0.014 inches, about 0.015 inches, about 0.016 inches, about 0.017 inches, about 0.018 inches, about 0.019 inches, and about 0.020 inches).

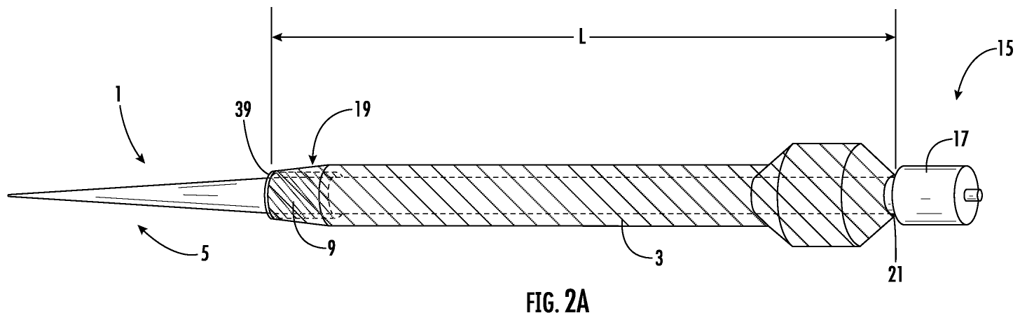

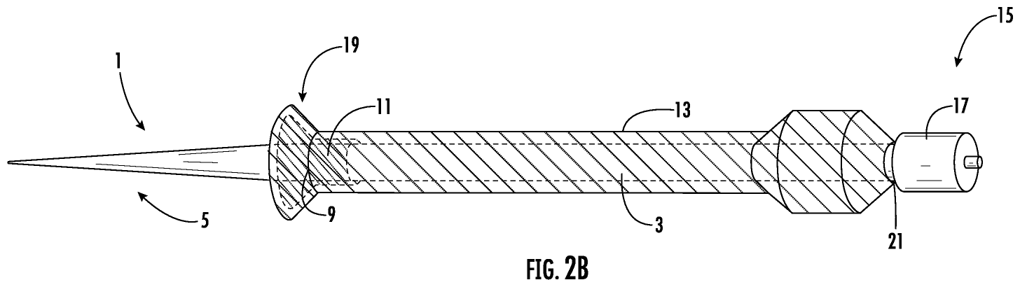

During insertion through sheath 13, expander 9 takes a compressed configuration as shown in FIG. 2A. In the compressed configuration, the expander 9 is compressed against the shaft 3 of the dilator 1. In an expanded configuration, the expander 9 extends radially away from the shaft 3 in one or more radial directions, as shown in FIG. 2B.

The dilator 1 also includes a proximal region 15 with a stopping feature 17. The stopping feature 17 is positioned a fixed distance from the expander 9. When inserting the dilator 1 into sheath 13, the stopping feature 17 indicates to the clinician that the dilator 1 is fully inserted and that the expander 9 is beneath the distal portion 19 of the sheath 13. The expander 9 is then prepared to expand the distal portion 19 of sheath 13 radially outward as in FIG. 2B, to prepare sheath 13 for medical device delivery. The stopping feature 17 can have a larger diameter than shaft 3 such that it abuts the proximal end 21 of sheath 13 as shown in FIGS. 2A and 2B. Alternatively, the stopping feature could be a visual indicator that the clinician aligns with proximal end 21 of the sheath 13, for example.

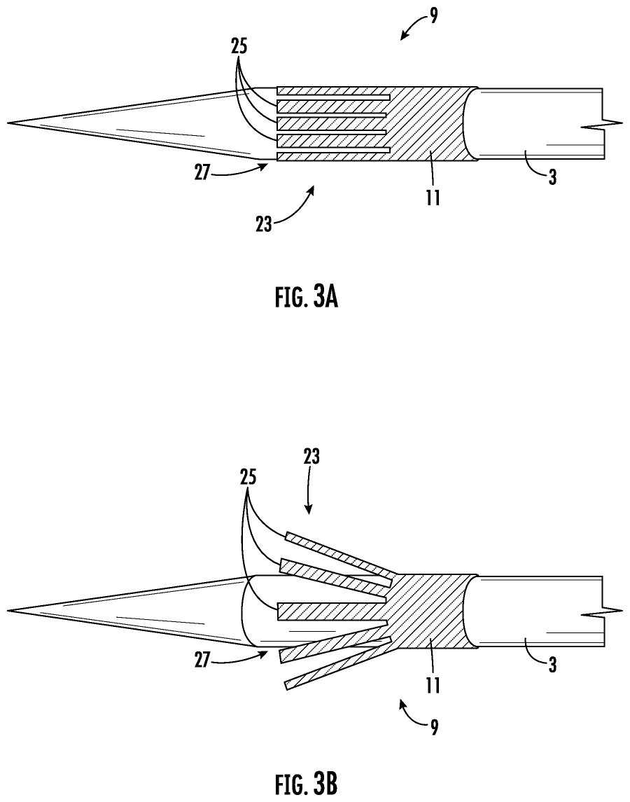

An example expander 9 is shown in FIGS. 3A and 3B. In this example, a tubular expander 9 is oriented parallel to the long axis of the shaft 3. The expander 9 wraps fully around the diameter of the shaft 3. The proximal side of the expander 9 includes continuous tubular portion 11, which is coupled to the shaft 3. After a length sufficient to securely bond tubular portion 11 to the shaft 3, the distal side 23 of expander 9 includes a plurality of longitudinally extending slits that extend proximally from the distal end 27 of the expander 9. The slits divide distal side 23 into a plurality of longitudinally extending arms 25 that can move away from shaft 3 to an expanded configuration. The diameter of the longitudinally extending arms 25 at the distal end 27 of expander 9 depends upon the length of the longitudinally extending slits—longer slits correspond to a larger diameter in the expanded configuration.

The longitudinally extending arms 25 of the expander 9 of FIGS. 3A and 3B move away from shaft 3 of the dilator 1 upon a change in the surroundings (a change in temperature or pH, for example). For example, in one examples, the expander 9 comprises a shape memory material (polymer or metal, such as, but not limited to, Nitinol). The shape memory material of the expander 9 will be configured to lie flat at room temperature. The expander 9 is sufficiently thin to not noticeably increase the diameter of the sheath 13 when lying underneath it. During prep, the clinician will insert the dilator 1 into the sheath 13 per standard practice. As the expander 9 is lying flat on the shaft 3, this step will not be compromised. Once ready for access, the clinician will track the dilator 1 and sheath 13 into the vessel per standard technique. Once the expander 9 warms sufficiently, the arms 25 hinge outward from the shaft 3 to create a larger diameter feature on the dilator 1, forcing the distal portion 19 of sheath 13 to open radially. If the expander 9 is made of metal, clinicians can potentially verify that the expander 9 has opened by checking on fluoroscopic imaging.

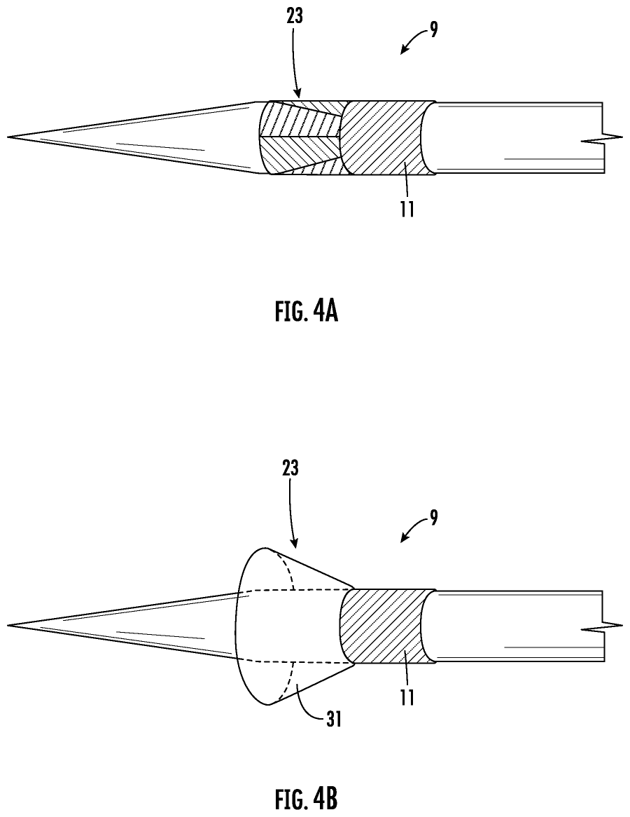

Another example expander 9 is shown in FIGS. 4A and 4B. Expander 9 includes proximal tubular portion 11 coupled to the shaft 3. The distal side 23 of the expander 9 is also continuous, but tapers up to a larger diameter. In the compressed configuration, the distal side 23 is folded down onto itself to remain tight to the shaft 3, creating one or more folds as shown in FIG. 4A. The folds unfold upon a change in the surroundings (a change in temperature or pH, for example), creating a funnel 31 as shown in FIG. 4B. For example, the expander 9 can be configured to change shape at body temperature, when the funnel 31 will force open the distal portion 19 of the sheath 13. The mechanism allows for the dilator 1 to be inserted into the sheath like normal, and subsequently inserted into the body following standard procedure, in an atraumatic manner. Once seated in the abdominal aorta, the expander will warm, opening the sheath tip, and allowing for easy insertion and retrieval through the tip for any subsequent interventional device tracked through the sheath.

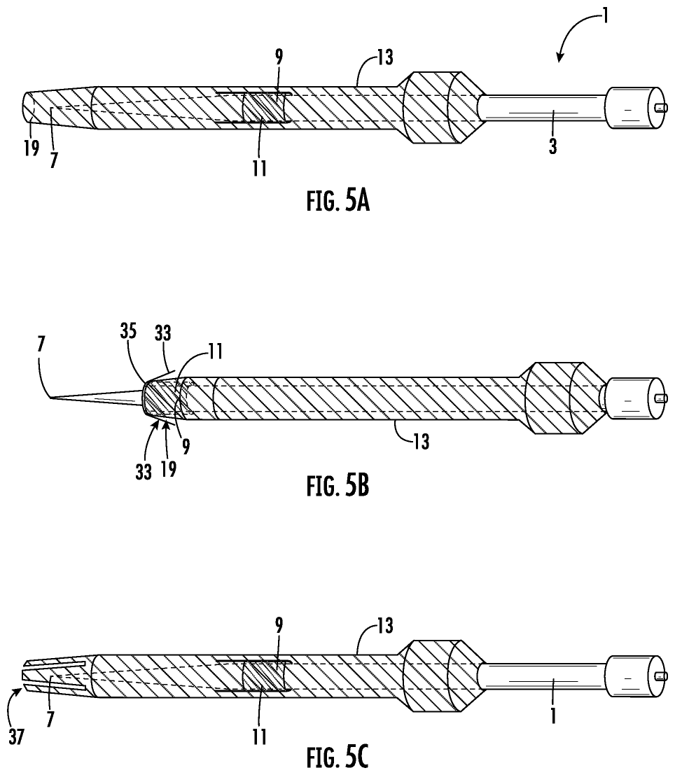

Another example expander 9 is shown in FIGS. 5A-5C. Here, the distal side of expander 9 includes one or more extensions 33 that are biased to extend radially away from shaft 3 and to point in a proximal direction. The expander 9 need not be formed of a shape memory material in this example, only a substance with ample strength. During insertion through sheath 13, the sheath 13 forces expander 9 into a compressed configuration, with extensions 33 tight against tubular portion 11 of the expander 9 as the dilator 1 advances axially within the sheath 13. The extensions 33 are positioned on the shaft 3 so that when the dilator 1 is fully inserted into the sheath 13, the extensions 33 extend just past the distal portion 19 of the sheath, allowing the expander 9 to take its expanded configuration as shown in FIG. 5B. The extensions 33 are thin enough to lay substantially flat against the sheath tip during insertion through the vasculature, not causing any trauma to the vessel.

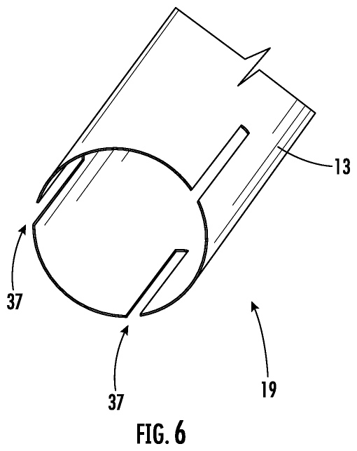

After tracking the sheath 13 to the desired location, the dilator 1 is removed from the sheath 13. As the user retracts the dilator 1 back through the sheath 13, the extensions 33 catch on the distal portion 19 of the sheath. The sheath 13 to be used in conjunction with the expander 9 in FIGS. 5A-5C has a distal portion 19 with two or more weakened longitudinal areas. The extensions 33 are sufficiently strong to tear open the sheath 13 in the areas where the distal portion 19 is weakened. The expander 9 splits the sheath distal portion 19 in the weakened longitudinal areas, leaving tears 37 that facilitate insertion and retrieval of interventional devices through the sheath distal portion 19 as shown in FIG. 5C and FIG. 6.

The location of the extensions 33 on the dilator 1 and the weakened regions on the distal portion 19 of sheath 13 can be rotationally aligned by providing alignment features or indicators on the proximal ends of sheath 13 and dilator 1. The alignment features or indicators could be visual, magnetic, audio, or tactile (for example, causing a beep or a shaking of the proximal ends when the alignment indicators meet). Alignment of the alignment features or indicators on the proximal ends of dilator 1 and sheath 13 corresponds to alignment of the extensions 33 and weakened regions at their respective distal ends.

After the extensions 33 are pulled through the weakened areas in the distal portion 19 of sheath 13 during dilator removal, they will encounter the fully formed wall of sheath 13. Each of the two or more extensions 33 comprises a corner or a hinge 35 coupling the extension 33 to the expander 9. The extensions 33 are able to rotate around their corners or hinges 35 to point toward the distal tip 7 of the dilator 1. The force on the extensions 33 as the expander 9 hits the main sheath wall is sufficient to cause the extensions 33 to hinge further outward. As the dilator is pulled back into sheath 13, the extensions 33 are forced to lay flat, facing towards dilator tip 7, hugged inwards by the inner lumen of sheath as shown in FIG. 5C.

The dilators disclosed herein can be made using the following methods. The shaft 3 is formed with a tapered distal region 5. The shaft can be formed, for example, by a single extrusion with the die tapering down to form the smaller, distal end. Alternatively, the shaft can be formed by multiple extrusions where a smaller extrusion forms the distal section of the dilator, which is joined to the larger proximal extrusion by means of using heat to melt the components together. An expander 9 is coupled to the shaft 3 adjacent to a distal tip 7 of the shaft 3. The expander 9 can be coupled to the shaft 3 by conventional techniques such as gluing, bonding or welding, or the expander 9 can be integrally formed with shaft 3. In some examples, the expander 9 is coupled proximal to the tapered distal region 5. A stopping feature 17 is coupled to a proximal region 15 of the shaft 3, a fixed distance from the expander 9. The stopping feature 17 can be coupled to the shaft 3 by conventional techniques such as gluing, bonding or welding, or the stopping feature 17 can be integrally formed with shaft 3. The stopping feature 17 can be added to the shaft 3 before or after the expander 9 is added to the shaft. Alternatively, one or both of the expander 9 and stopping feature 17 can be integrally formed with the shaft 3 of the dilator 1. In some examples, the stopping feature 17 can be formed to have a larger diameter than the shaft 3. In other examples, the stopping feature is a visual indicator that is marked directly on the shaft 3.

As shown in FIB. 3A, tubular portion 11 of expander 9 can be coupled such that extends circumferentially around the shaft 3. In some examples, expander 9 is formed of a shape memory material. The shape memory material may be cut with longitudinally extending slits that extend proximally from distal end 27 of the expander 9. The longitudinally extending slits may be cut to a desired length using lasers or mechanical cutting tools, for example. The longitudinally extending slits separate the expander 9 to form longitudinally extending arms 25.

The expander 9 undergoes a pretreatment process to set it to the compressed configuration. The shape memory material causes the expander 9 to transform to the expanded configuration upon a change in surroundings (such as temperature or pH, for example), causing longitudinally extending arms 25 to expand radially outward, as seen in FIG. 3B.

In some examples, expander 9 is formed of a shape memory material with a funnel on the distal side 23 of the expander 9. The methods of making further include folding the funnel into a tubular shape, as shown in FIG. 4A. The expander 9 is set to this compressed configuration in a pretreatment process. Upon a change in surroundings (such as a change in temperature or pH, for example), the distal side 23 of expander 9 unfolds to the funnel 31 shown in FIG. 4B.

In some examples, the expander 9 is formed to include two or more extensions 33, as shown in FIGS. 5A-5C. The expander 9 with extensions 33 can be formed by molding or machining. The extensions 33 are biased to point in a proximal direction. The extensions 33 are also biased to extend radially away from other portions of the expander 9, such as tubular portion 11 and ultimately, the shaft 3 of dilator 1 once the expander 9 is attached. The extensions 33 are thin enough to not be raised sufficiently above the rest of the dilator surface (less than 0.015 inches), but sufficiently strong enough to resist loading from the sheath during insertion and removal of the dilator into the sheath (greater than about 30 Newtons, for example). The extensions 33 are coupled to the expander 9 at a corner or hinge 35, which allow the extensions 33 to flex 180 degrees to point in a distal direction as shown in FIG. 5C. Distal portion 19 of the sheath 13 can be weakened to facilitate tearing by the extensions 33 as the dilator 1 is removed from the sheath 13. For example, the distal portion 19 can be scoured or laser etched to create weakened longitudinal areas with thinner walls than the surrounding areas of the sheath 13. In some examples, to create weakened areas in the sheath 13, the wall of the sheath is cut completely through. The cut is then reflowed back together at a lower temperature than for the rest of the sheath forming. This creates a weakened area that separates more easily from the other areas of the sheath. The cut can be made manually, or by laser cutting, or by a stamping die, for example.

Treatment methods include first inserting the tapered dilator 1 into a sheath 13. In some examples, the dilator 1 is translated axially until a stopping feature 17 on the dilator 1 is a predetermined fixed distance L from the distal end 39 of the sheath 13, as shown in FIG. 2A. For example, the stopping feature 17 abuts proximal end 21 of sheath 13 when distal end 39 is the fixed distance L from the stopping feature 17. When the stopping feature 17 is the predetermined fixed distance L from the sheath distal end 39, at least part of the expander 9 is positioned beneath distal portion 19 of the sheath 13. In some examples, the tapered dilator 1 is axially translated within sheath 13 until at least part of expander 9 is distal to the distal portion 19 of sheath 13. For example, in FIG. 5B, extensions 33 are positioned distal to the distal portion 19 of sheath 13.

The dilator 1 and sheath 13 together are inserted into the vasculature of the subject. Once the sheath 13 is properly positioned within the vasculature, distal portion 19 of sheath 13 is expanded, or torn, using expander 9. In some examples, the expander 9 is induced to take the expanded configuration beneath the distal portion 19 of the sheath 13 (for example by a change in the surroundings). The expander 9 can take an expanded configuration, for example, by moving longitudinally extending arms 25 radially away from the shaft 3 of the dilator 1, as shown in FIG. 3B. Alternatively, the expander 9 can take an expanded configuration by unfolding one or more folds of the expander 9 to create a funnel shape, as shown in FIG. 4B. Alternatively, extensions 33, which are positioned distal to the distal portion 19 of the sheath 13 as shown in FIG. 5B, can tear the distal portion 19 of the sheath 13 as the dilator 1 is removed from the sheath. The sheath 13 can be provided with weakened areas extending along the distal portion 19, and the extensions 33 of expander 9 can be rotationally aligned to these weakened portions to facilitate tearing of the distal portion 19 of the sheath 13. As the dilator 1 is pulled proximally through sheath, the extensions 33 rotate around corners or hinges 35, transforming the expander 9 from a natural state with extensions 33 pointing in a proximal direction to an inverted state with extensions 33 pointing in the distal direction as shown in FIG. 5C.

Once the dilator 1 is removed from sheath 13, a medical device can be delivered through the sheath 13. The medical device passes through distal portion 19 with relative ease due to the previous expansion or tearing of the distal portion 19 of the sheath 13.

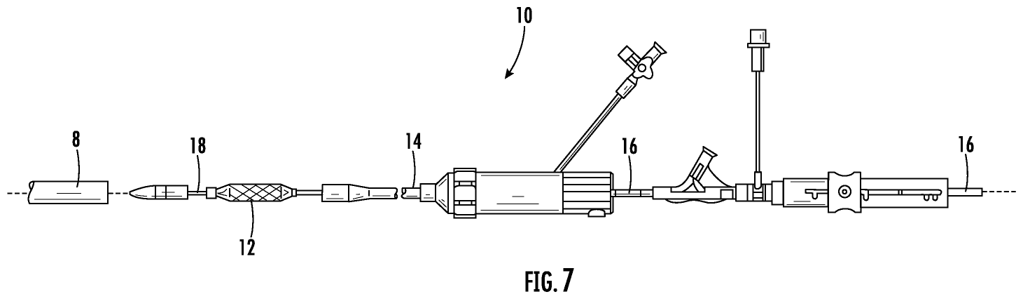

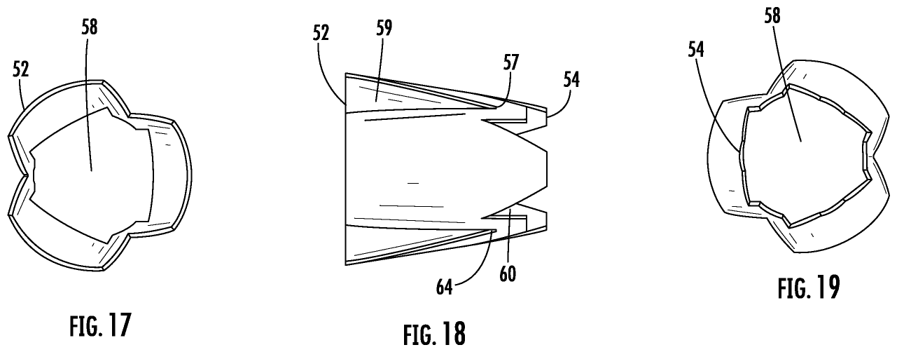

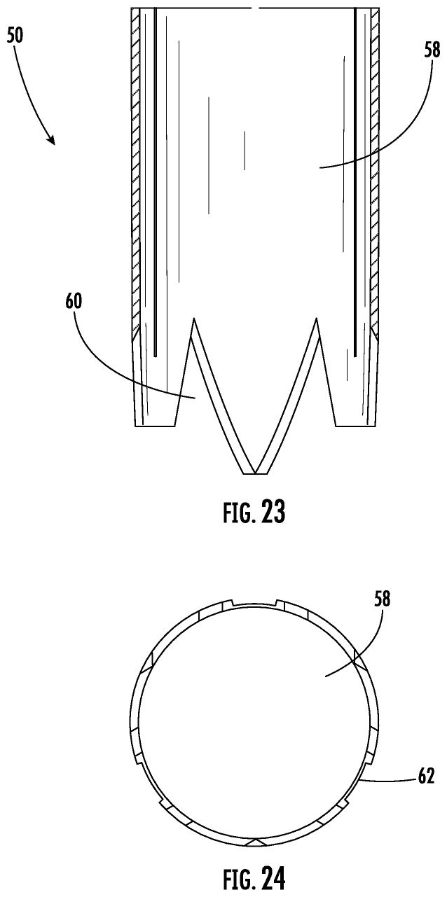

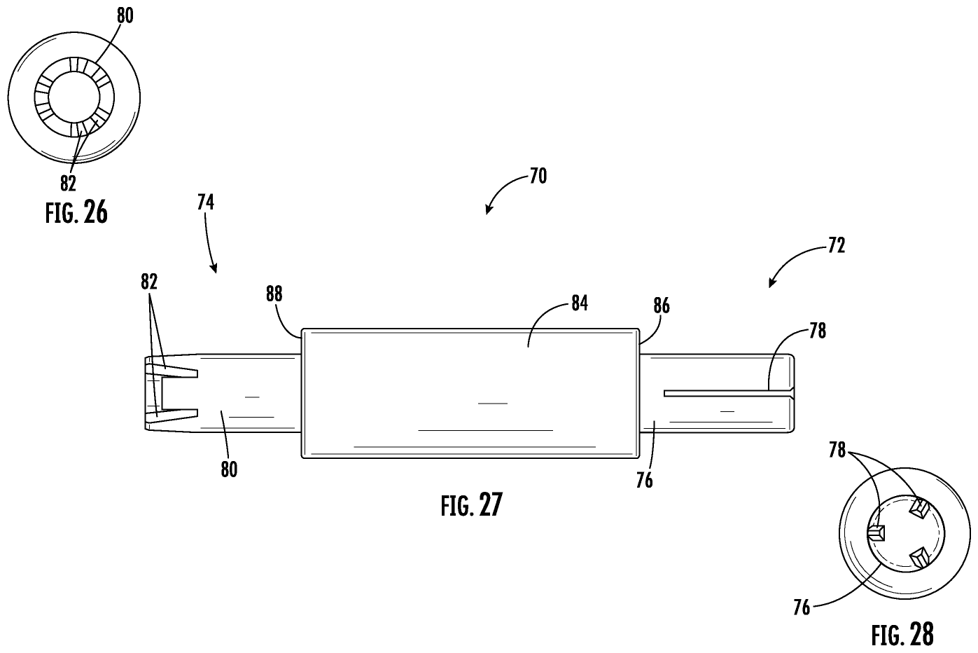

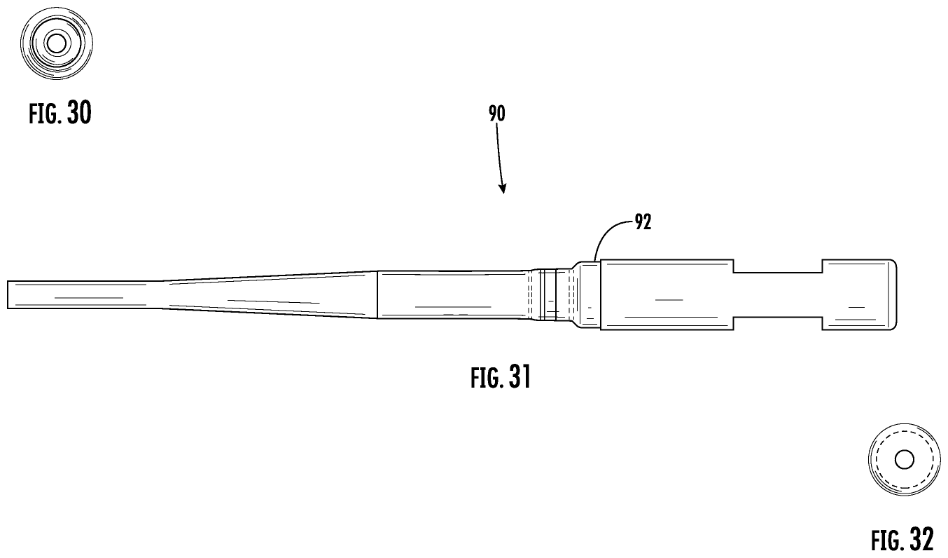

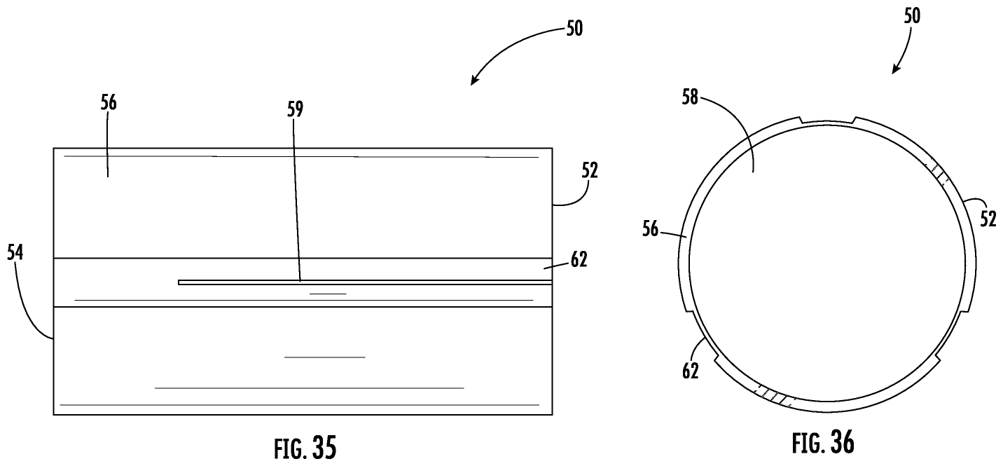

Furthermore, as noted above, a sheath can be used to promote the smooth transition from the sheath to a proximally positioned sheath hub, while also providing a seal between the sheath and the sheath hub. During manufacturing, the proximal end of the sheath must be enlarged and securely and reliably coupled to the sheath hub. This disclosure contemplates improved manufacturing methods to reduce assembly and processing time while improving engagement between the sheath and the sheath hub. Disclosed herein in reference to FIGS. 7-37 are examples of a sheath including a flared outer sleeve coupled to the proximal end of the sheath proximate the coupling with the sheath hub. As described in more detail below, the outer sleeve facilitates a secure and sealed connection between the sheath body and the sheath hub. Methods are also disclosed that optimize uniform expansion, circularity, and evenness of reflowed material during manufacture of the combined sheath and outer sleeve.

FIG. 7 illustrates an exemplary sheath 8 in use with a representative delivery apparatus 10, for delivering an implant 12, or other type of implantable, to a patient. The delivery apparatus 10 can include a steerable guide catheter 14 (also referred to as a flex catheter) and a balloon catheter 16 extending through the guide catheter 14. The guide catheter 14 and the balloon catheter 16 in the illustrated example are adapted to slide longitudinally relative to each other to facilitate delivery and positioning of the implant 12 at an implantation site in a patient's body, as described in detail below. A similar delivery apparatus 10 can be used with the dilator 1 and sheath 13 described above.

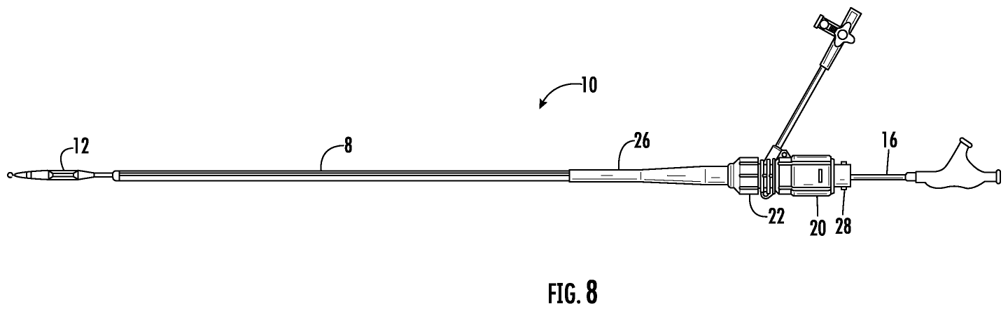

FIG. 8 illustrates the sheath 8 of FIG. 7. The sheath 8 is an elongate, expandable tube that can include a hemostasis valve at the proximal end 32 of the sheath to stop blood leakage. As illustrated in FIG. 8, the sheath assembly can comprise the sheath hub 20 at a proximal end of the device and an expandable sheath 8 extending distally from the sheath hub 20. The sheath hub 20 can function as a handle for the device. The expandable sheath 8 has a central lumen to guide passage of the delivery apparatus for the medical device/prosthetic heart valve. The sheath 8 includes a seal tube 26/strain relief portion. The seal tube 26 is coupled to the distal end of the sheath hub 20 and creates a smooth transition surface between the sheath 8 and the sheath hub 20. The frustoconical seal tube 26 body has a proximal end and a distal end and a central lumen extending longitudinally therethrough. The seal tube 26 tapers from the proximal end to the distal end such that the diameter of the seal tube 26 at the proximal end is greater than the diameter of the seal tube 26 at the distal end of the seal tube 26.

The sheath 8 can have a natural, unexpanded outer diameter that will expand locally upon passage of the medical device. Expandable sheaths, formed of highly elastomeric materials, allow for the dilating of the vessel to be performed by the passing prosthetic device. Expandable sheaths are disclosed in PCT Application No. PCT/US2021/050006 (corresponding to U.S. Provisional Application No. 63/091,226, filed Oct. 13, 2020), entitled “Expandable Sheath Including Reverse Bayonet Locking Hub”, U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. application Ser. No. 14/880,109, entitled “Expandable Sheath,” U.S. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. application Ser. No. 15/997,587, entitled “Expandable Sheath for Introducing an Endovascular Delivery Device into a Body,” U.S. application Ser. No. 16/378,417, entitled “Expandable Sheath,” the disclosures of which are herein incorporated by reference.

In certain aspects, the expandable sheath 8 can comprise a plurality of coaxial layers extending along at least a portion of the length of the sheath 8. For example, as will be described in more detail below, the sheath 8 includes an outer sleeve 50 is coupled to an elongated inner sleeve 40 to provide a secure and sealed connection between the sheath 8 and the sheath hub 20. The outer sleeve 50 is coupled to the proximal end 42 of the inner sleeve 40. and creates a transition surface between the elongated inner sleeve 40 and the sheath hub 20.

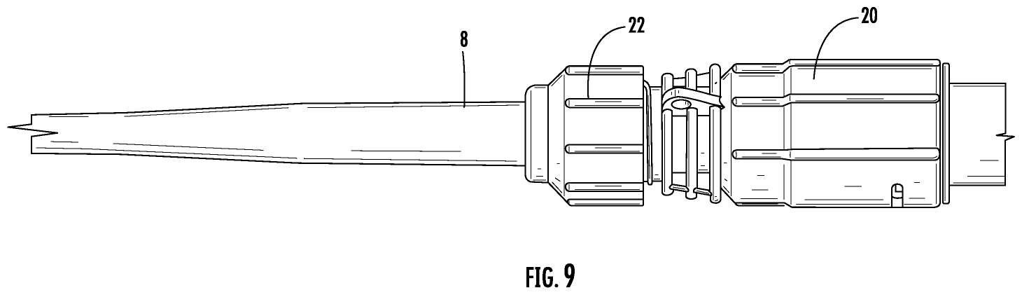

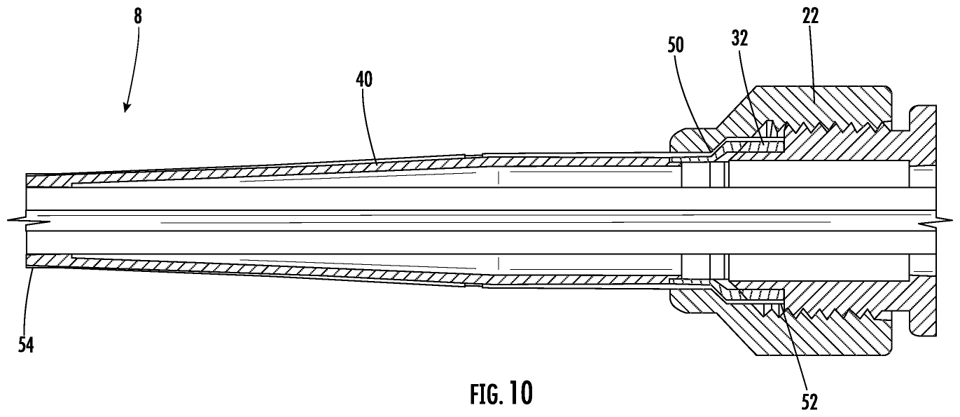

The examples disclosed herein provide a combined sheath 8 (inner sleeve 40 and outer sleeve 50) that has a uniform thickness about the proximal end 32 of the sheath 8. The uniform thickness provides a secure body that couples the sheath 8 to the sheath hub 20. The secure body prevents the sheath 8 from separating from the sheath hub 20 during use by providing a uniform interference fit about the outer diameter sheath 8 at the sheath hub 20 and the inner diameter of a sheath hub cap 22. Further, as described below, the combined sheath 8 (elongated inner sleeve 40 and outer sleeve 50) is pre-expanded/flared at the proximal end to promote engagement between the sheath 8 and the sheath hub 20. As illustrated in FIGS. 9-10, the sheath 8 tapers along the outer sleeve 50, from the proximal end 52 to the distal end 54 of the outer sleeve 50, such that the diameter of the sheath 8 at the proximal end 52 of the outer sleeve 50 is greater than the diameter of the sheath 8 at the distal end 54 of the outer sleeve 50.

FIGS. 7-11 show the combined sheath 8 including the elongated inner sleeve 40 and an outer sleeve 50. As will be described in more detail below, a portion of the of the inner sleeve 40 is disposed in a central opening 58 of the outer sleeve 50. The inner sleeve 40 and the outer sleeve 50 are coupled together forming a coupled proximal end 32 of the sheath 8 that has a uniform diameter about its circumference.

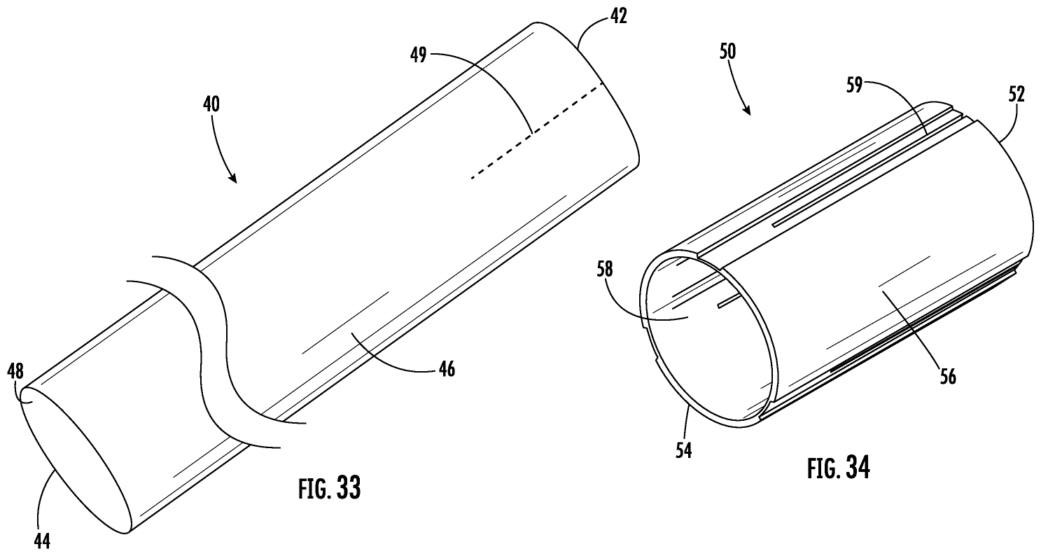

As provided in FIG. 12, the inner sleeve 40 is a tubular body that has a flared proximal end 42, a distal end 44, and an inner sleeve body 46 that extends between the proximal end 42 and the distal end 44. The inner sleeve body 46 defines a central opening 48 defining a lumen extending through the inner sleeve 40 between the proximal and distal ends 42, 44. The flared proximal end 42 is formed by slits 49 and/or openings extending from the proximal end 42 of the inner sleeve 40 along the inner sleeve body 46. The inner sleeve 40 includes three circumferentially spaced slits 49 that extend from the proximal end 42 along a portion of the inner sleeve body 46. The slits 49 are uniformly/evenly spaced around the circumference of the inner sleeve 40. In other implementations the inner sleeve 40 includes one, two, four, five or any other number of slits 49 suitable to allow the inner sleeve 40 to expand outwardly. Each of the plurality of longitudinal slits 49 of the inner sleeve 40 extend along a line generally parallel with a longitudinal axis of the inner sleeve 40 (see FIG. 33). In some implementations, at least one of the slits 49 of the inner sleeve 40 extend in a direction that is not parallel with the longitudinal axis of the inner sleeve 40.

The slits 49 extend along a length of the inner sleeve body 46 sufficient to provide a structurally rigid proximal end 42 that is securable to a sheath hub 20. As illustrated in FIG. 12, each of the plurality of longitudinal slits 49 has a same length along the inner sleeve 40. In other examples, at least one of the longitudinal slits 49 of the inner sleeve 40 has a length that is different from at least one of the other slits 49 of the inner sleeve 40.

In some examples, the slits 49 of the inner sleeve 40 extend from 0.36 inches to 0.54 inches along the inner sleeve body 46. For example, the slits 49 of the inner sleeve 40 extend 0.450 inches along the inner sleeve body 46. In other implementations, the slits 49 of the inner sleeve 40 extend from 0.50 inches to 0.60 inches along the inner sleeve body. For example, the slits 49 of the inner sleeve 40 extend 0.50 inches along the inner sleeve body 46.

The slits 49 define separated portions of the inner sleeve body 46 that are independently expandable in separate directions as illustrated in FIG. 12. As the proximal end 42 of the inner sleeve 40 expands, the spacing between the separated portions increases along/at the slits 49 forming a tapered or flared proximal end. The separated portions are outwardly bendable away from a central axis of the inner sleeve 40 and form a larger opening diameter at the proximal end 42 than the distal end 44.

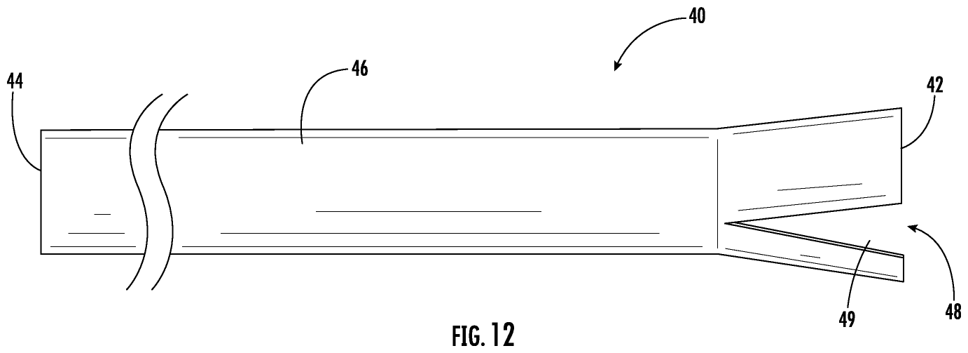

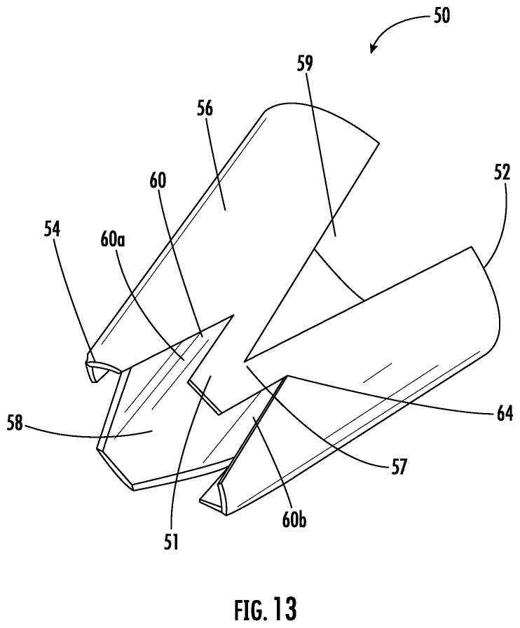

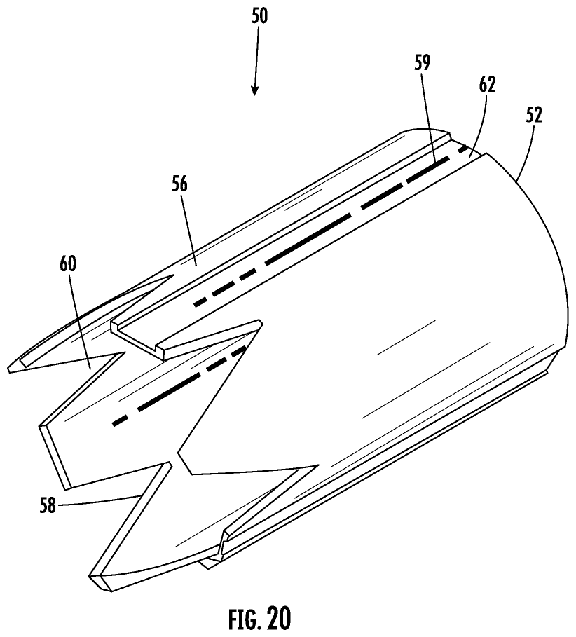

As described above in reference to FIGS. 9-11, an outer sleeve 50 is provided over the proximal end 42 of the inner sleeve 40 and the distal end of the sheath hub 20. As illustrated in FIG. 13-24, the outer sleeve 50 includes a tubular-shaped outer sleeve body 56 that extends between a proximal end 52 and a tapered distal end 54. The outer sleeve body 56 defines a central opening 58 defining a lumen extending between the proximal and distal ends 52, 54. The flared proximal end 52 of the outer sleeve 50 is formed by slits 59 and/or openings extending from the proximal end 52 along a portion of the outer sleeve body 56. For example, as illustrated in FIGS. 13-19, the outer sleeve 50 includes three circumferentially spaced slits 59 that extend from the proximal end 52 along a portion of the outer sleeve body 56. In other implementations, the outer sleeve 50 includes one, two, four, five or any other number of longitudinal slits 59 suitable to allow the outer sleeve 50 to flare. Each of the plurality of longitudinal slits 59 of the outer sleeve 50 extend along a line generally parallel with a longitudinal axis of the outer sleeve 50 (see FIGS. 20, 21, 34). In some implementations, at least one of the slits 59 of the outer sleeve 50 extend in a direction that is not parallel with the longitudinal axis of the outer sleeve 50. Each of the plurality of longitudinal slits 59 of the outer sleeve 50 has a same length along the outer sleeve 50. In some implementations, at least one of the longitudinal slits 59 of the outer sleeve 50 has a length that is different from at least one of the other slits 59 of the outer sleeve 50.

In some examples, the slits 49 of the inner sleeve 40 and/or the slits 59 on the outer sleeve 50 extend from 0.360 inches to 0.540 inches or any other length suitable to provide a structurally rigid proximal end that is securable to a sheath hub 20. In other implementations, the slits 49 of the inner sleeve 40 and/or the slits 59 on the outer sleeve 50 extend from 0.400 inches to 0.500 inches along the outer sleeve body. For example, the slits 49 of the inner sleeve 40 and/or the slits 59 on the outer sleeve 50 extend 0.450 inches along the outer sleeve body 56.

The slits 59 define separated portions of the outer sleeve body 56 that are independently expandable in separate directions. As illustrated in FIGS. 13-19, as the proximal end 52 of the outer sleeve 50 expands, the spacing between the separated portions increases along/at the slits 59 forming a tapered or flared proximal end. The separated portions are outwardly bendable away from a central axis of the outer sleeve 50 and form a larger opening diameter at the proximal end 52 than the distal end 54.

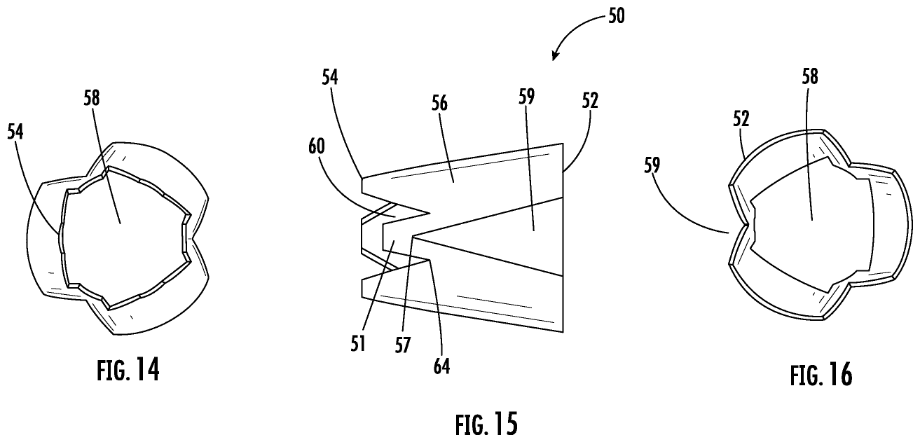

As illustrated in FIGS. 13-24, the outer sleeve 50 includes window cuts 60 formed at the distal end 54 and extend along a length of the outer sleeve body 56. The window cuts 60 form separated portions of the distal end 54 of the outer sleeve 50. The separated portions are independently bendable/movable in separate directions. The window cuts 60 have a generally decreasing shape/width such that the spacing between opposing edges of adjacent portions decreases along the outer sleeve body 56. In the example shown in FIG. 13 (and FIGS. 15, 18, 20, 21, 23), the window cuts 60 have a proximally extending triangular shape. As such, the separated portions bend inwardly and/or circumferentially toward each other such as the opposing edges of the adjacent portions move together. As a result, the outer sleeve body 56 defines a decreasing taper between at least a longitudinal midline of the outer sleeve body 56 and the distal end 54. In other implementations the window cuts 60 are rectangular shaped, slits, or any other regular or irregular shape suitable to allow the tube to contract radially.

As provided in FIG. 13, outer sleeve 50 includes window cuts 60 that extend from the distal end 54 of the outer sleeve 50 along a portion of the outer sleeve body 56. In the example outer sleeve 50 illustrated in FIG. 13 (and FIGS. 14-21), the outer sleeve 50 includes six window cuts 60 extending proximally from the distal end 54 along a portion of the length of the outer sleeve body 56. In other examples, the outer sleeve 50 includes any other number of window cuts 60 suitable to allow the outer sleeve 50 to flare or taper inwards.

The window cuts 60 are circumferentially spaced apart from the slits 59 around the outer sleeve 50. For example, each slit 59 is circumferentially centered between an adjacent pair of window cuts 60 around the outer sleeve 50 as shown in FIG. 15. As such, there are two window cuts 60 for each slit 59 in the outer sleeve 50. The window cuts 60 are circumferentially evenly spaced apart to either side of each slit 59. The window cuts 60 are evenly spaced apart to either side of a corresponding slit 59 by a circumferential distance from 0.064 inches and 0.096 inches. For example, window cuts 60 are evenly spaced apart to either side of a corresponding slit 59 by 0.080 inches. Similarly, the proximal end 64 of each window cut 60 is spaced from a corresponding adjacent slit 59 by a circumferential distance from 0.064 inches to 0.130 inches.

The window cuts 60 have an axial length from 0.150 inches to 0.250 inches measured axially/longitudinally along the outer sleeve 50. In an example, the window cuts 60 have an axial length of 0.200 inches. The window cuts 60 have a circumferential width from 0.070 inches to 0.120 inches measured at the distal end 54 of the outer sleeve 50. In an example, the window cuts 60 have circumferential width of 0.090 inches around the distal end 54 of the outer sleeve 50.

In the implementations shown in FIGS. 13-24, the length of each slit 59 is greater than the length of each window cut 60. This allows the distal end 54 of the outer sleeve 50 to compress and the proximal end 52 of the outer sleeve 50 is expanded, forming a cone shape. In other implementations, the length of each slit 59 is less than or equal to the length of each window cut 60. In these examples, as the proximal and distal ends of the outer sleeve contract and expand respectively, the outer sleeve 50 forms an irregular tapered structure where the diameter of the proximal end 52 is less than the diameter of the distal end 54.

In some examples, a portion each slit 59 axially overlaps with a portion of the corresponding window cuts 60 around the circumference of the outer sleeve 50. For example, as illustrated in FIG. 13, outer sleeve 50 includes a slit 59 circumferentially centered between two corresponding proximally extending triangular shaped window cuts 60a, 60b. A segment 51 of the outer sleeve 50 extends between the trailing edge of the first window cut 60a and the leading edge of the second window cut 60b. As provided in FIG. 13, the length of the slit 59 extends along the outer sleeve body 56 and axially overlaps with the length of the window cuts 60a, 60b (see also, FIGS. 15, 18, 20, 21). The slit 59 extends into the segment 51 such that the distal end point 57 of the slit 59 is located distal the proximal end point 64 of the window cuts 60a, 60b. In further examples, at least one of the slits 59 is of a length that does not extend beyond the proximal end 64 of the corresponding adjacent window cuts 60.

As illustrated in FIG. 15, the distal end surface of segment 51 does not extend to the distal end 54 of the outer sleeve body 56. The distal end surface of segment 51 extends generally parallel with the distal end 54 of the outer sleeve body 56. In other examples, the distal end surface of segment 51 extends in a direction transverse to the distal end 54 of the outer sleeve body 56. In further examples, the distal end surface of the segment 51 extends to the distal end 54 of the outer sleeve body 56.

In some examples, the outer sleeve 50 includes recesses 62 that extend along all or a portion the length of outer sleeve 50. An example outer sleeve 50 including recesses 62 is illustrated in FIGS. 20-24. The recesses 62 are provided to retain reflow material during the reflow process as will be described in more detail below. The width of each recess 62, in part, determines the thickness of the reflow material retained at each recess 62 location. The width of each recess 62 is inversely related to the thickness of the reflow material retained at the recess 62. As such, a wider recess allows the reflow material to spread and form a thin layer of reflow material relative to a more narrow recess 62, which would retain a thicker layer of reflow material.

The recesses 62 extend between the proximal end 52 of the outer sleeve 50 and the distal end 54 of the outer sleeve 50. In other examples, one or more of the recesses 62 extend along a portion of the length of the outer sleeve 50. Each of the recesses 62 extend circumferentially from either side of each of the longitudinal slits 59. For example, the recesses 62 extend from 0.030 inches to 0.070 inches from either side of each of the slits 59 or any other distance suitable to control accept a desired amount of reflow material that provides a desired material thickness. In some examples, the recesses 62 extend 0.040 inches from either side of each of the slits 59. In further examples, the recesses 62 extend 0.056 inches from either side of each of the slits 59.

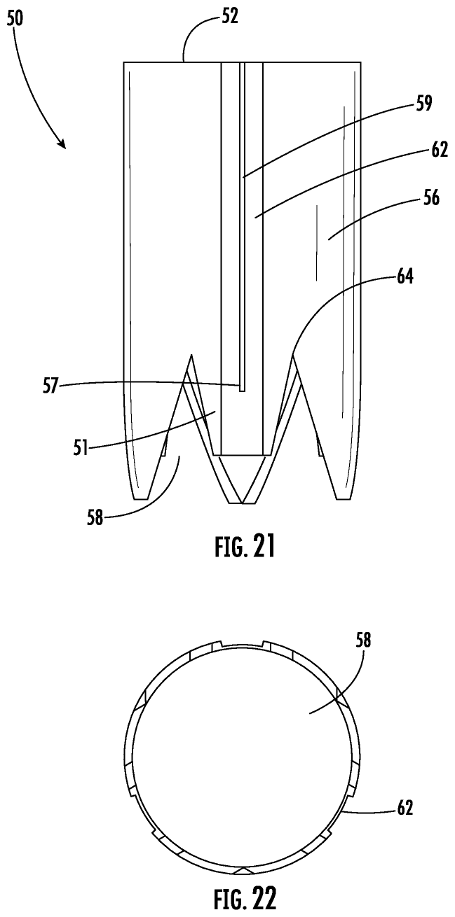

In the example sleeve illustrated in FIGS. 20-24, the outer sleeve 50 includes three recesses 62 extending along the entire length of the outer sleeve 50. The recesses 62 extend radially from an outer surface of the outer sleeve 50 in a direction toward the central opening 58. As illustrated in FIGS. 22 and 24, the height of the recess is defined as the distance from the outer surface of the outer sleeve 50 to the circumferential surface of the recess 62. In some examples, the recesses 62 have a height greater than half of the thickness of the outer sleeve 50. In other examples, the height of the recesses 62 is equal to or less than the thickness of the outer sleeve 50. As provided in FIGS. 22 and 24, the recesses 62 include squared off or rectilinear leading and trailing circumferential edges. That is the circumferential surface of the recess 62 extends circumferentially between leading and trailing edges. In FIGS. 22 and 24, the leading and trailing edges are shown as extending in a radial direction from the center point of the outer sleeve 50 from the circumferential surface to the outer surface of the sleeve. In other examples, the leading and trailing edges extend at an angle with respect to the circumferential surface of the recess 62 providing tapered leading and trailing edges.

As described above, the outer sleeve 50 and the inner sleeve 40 are coupled together to form the coupled proximal end 32 of the sheath 8 as shown in FIGS. 9-11. During assembly, the slits 49, 59 and window cuts 60 are cut into the inner sleeve 40 and outer sleeve 50. In other examples, the inner sleeve 40 and outer sleeve 50 come pre-cut. A portion of the proximal end 42 of the inner sleeve 40 is disposed in the central opening 58 of the outer sleeve 50 such that the proximal end 42 of the inner sleeve 40 and the proximal end 52 of the outer sleeve 50 are longitudinally aligned. The slits 59 of the outer sleeve 50 are circumferentially spaced apart from the slits 49 of the inner sleeve 40 and disposed in a flared position. The inner sleeve 40 and the outer sleeve 50 form the coupled proximal end 32, by undergoing reflow heat treatment. As such, the openings of the slits 49 of the inner sleeve 40 are filled by the outer sleeve body 56 and the openings of the slits 59 and window cuts 60 of the outer sleeve 50 are filled by the inner sleeve body 46, thereby forming a uniform circumference at the coupled proximal end 32.

In the example sheath shown in FIGS. 9-24, the number of slits 49 in the inner sleeve 40 is equal to the number of slits 59 in the outer sleeve 50. FIGS. 12-24 show three slits 49 in the inner sleeve 40 and three slits 59 in the outer sleeve 50. In other implementations, the inner sleeve 40 and the outer sleeve 50 each have a different number of longitudinal slits 59 suitable to form a desired shape at the coupled proximal end 32.

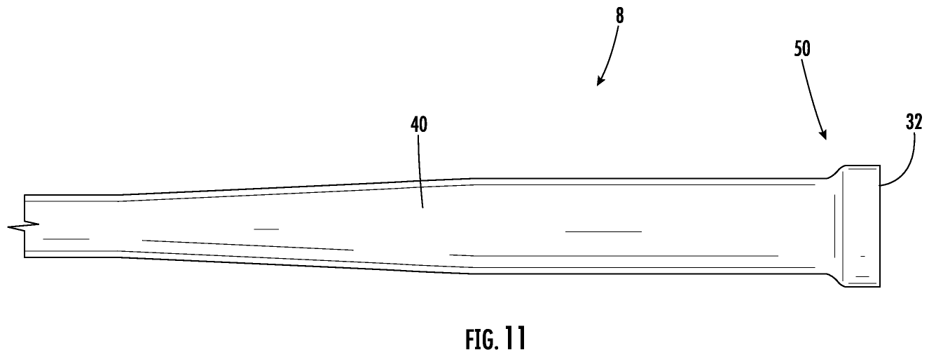

In the examples shown in FIGS. 10-11, the coupled proximal end 32 has a flared diameter, which includes a fillet, rounded, and/or tapered transition surface between body of the sheath 8 and the flared, larger diameter proximal end. In other implementations, the proximal end forms a sharp, rectilinear transition surface or any other shape surface suitable to couple to a sheath hub 20.

The inner sleeve 40 and the outer sleeve 50 each have a same thickness, such that the uniform thickness of the combined outer sleeve 50 and the inner sleeve 40 is double the thickness of the inner sleeve and/or outer sleeve 50. The inner sleeve 40 thickness and the outer sleeve 50 thickness can range from 0.006 inches to 0.016 inches, or any thickness suitable to provide a body that is securely couplable to a sheath hub 20. For example, the thickness of each of the inner sleeve 40 and the outer sleeve 50 is from 0.010 inches to 0.012 inches. In a further example, the thickness of each of the inner sleeve 40 and the outer sleeve 50 is 0.010 inches. In other examples, the inner sleeve 40 and outer sleeve 50 have different thicknesses. The inner sleeve 40 thickness and/or outer sleeve 50 thickness can range from 0.006 inches to 0.016 inches.

The inner sleeve 40 and outer sleeve 50 are each formed from the same material. For example, the inner sleeve 40 and the outer sleeve are each formed from High Density Polyethylene (HDPE). In other implementations, the inner sleeve 40 and the outer sleeve are each formed from HDPE, Polypropylene, a mixed Polymer material or any other material suitable for forming a sheath. In other implementations the inner sleeve 40 and the outer sleeve 50 are formed from materials different from each other. For example, the inner sleeve 40 can be formed from HDPE while the outer sleeve 50 is formed from Polypropylene.

As described above, the combined inner sleeve 40 and outer sleeve 50 is couplable to a sheath hub 20 and a sheath hub cap 22 (FIGS. 8-10). As shown in the cross section view of FIG. 10, the proximal end 32 of the sheath 8 is sandwiched between the sheath hub 20 and the sheath hub cap 22. The sheath hub cap 22 secures the coupled proximal end 32 of the sheath 8 to the sheath hub 20 by an interference fit. In other implementations, the sheath 8 is coupled to the sheath hub 20 by adhesive or any other coupling mechanism suitable for coupling a sheath 8 to a sheath hub 20.

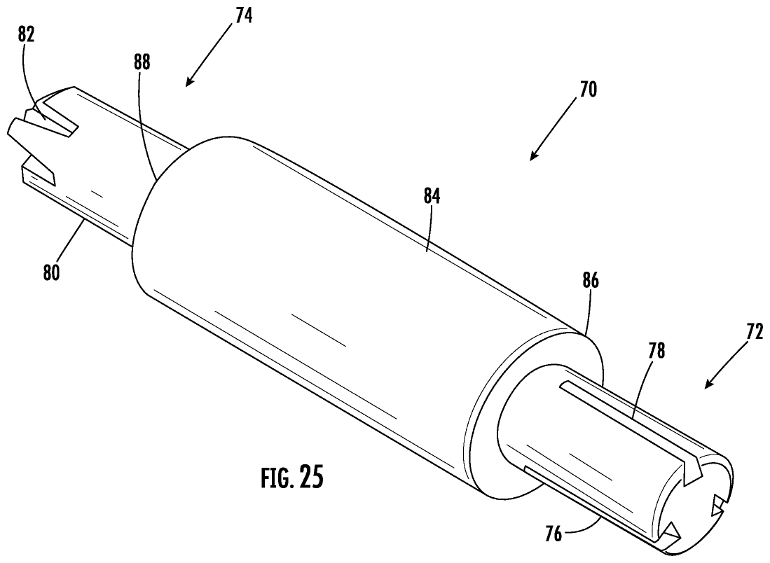

FIGS. 25-28 shows a cutting guide device 70 that can be used to form the longitudinal slits 49, 59 and the window cuts 60 in the inner sleeve 40 and outer sleeve 50. The cutting guide 70 includes a slit cut guide 72, a window cut guide 74, and a central body 84 coupled between the slit cut guide 72 and the window cut guide 74. The central body 84 of the cutting guide device 70 has a cylindrical body that has a first end 86, a second end 88 opposite and spaced apart from the first end 86. The slit cut guide 72 is coupled to the first end 86 of the central body 84, and the window cut guide 74 is coupled to the second end 88 of the central body 84. While the central body is shown as having a cylindrical shape, in other implementations, the central body 84 is cuboid or any other shape suitable to provide a central body 84 that can be manipulated by a user that is cutting a sheath 8.

The slit cut guide 72 includes a first cylindrical body 76, which has an outer edge and three longitudinal grooves 78 circumferentially disposed about the first cylindrical body 76. The longitudinal groves 78 extend from the outer edge and along a portion of the slit cut guide 72. Each of the of longitudinal grooves 78 define a guide channel shaped to secure and/or receive a cutting tool such as a blade or laser cutter to form a longitudinal slit in a tube (e.g., the inner sleeve 40 and/or outer sleeve 50) disposed about the slit cut guide 72. Each longitudinal groove 78 has an axial length of ranging from 0.600 inches to 0.400 inches, or any other length suitable to guide a cutting tool to form a desired longitudinal slit cut in an inner sleeve 40 or an outer sleeve 50. For example, the longitudinal groove 78 has an axial length of 0.500 inches. The longitudinal groove 78 can have a depth ranging from 0.016 inches to 0.024 inches. For example, the longitudinal groove 78 can have a depth of 0.020 inches. In other examples, the longitudinal groove 78 can have a depth ranging from 0.040 inches to 0.060 inches. For example, the longitudinal groove 78 can have a depth of 0.050 inches. While the example illustrated in FIGS. 25-28 includes three longitudinal grooves 78, it is contemplated that the slit cut guide 72 can include two grooves, four grooves, or any number of grooves suitable to guide a cutting tool to form a desired number of longitudinal slits 49, 59 in the inner sleeve 40 and/or an outer sleeve 50.

The window cut guide 74 includes a second cylindrical body 80 that has an outer edge and six V-shaped and/or angled grooves 82 circumferentially disposed about the second cylindrical body 80. The V-shaped groves 82 extend from the outer edge and along a portion of the window cut guide 74. Each of the plurality of V-shaped grooves 82 define guide edges to secure and/or receive a cutting tool (e.g., a blade) to form a window cut 60 in a tube (e.g., outer sleeve 50) disposed about the window cut guide 74. The axial length of each V-shaped groove 82 ranges from 0.160 inches to 0.240 inches. For example, the length of the V-shaped groove 82 is 0.200 inches. The width of each V-shaped grove 82 ranges from 0.100 inches to 0.150 inches. For example, the width of each V-shaped grove 82 is 0.125 inches. In other implementations, the V-shaped grooves 82 have any axial length from 0.150 inches to 0.250 inches, and a circumferential width from 0.0850 inches to 0.150 inches, or any other length and circumferential width suitable guide a cutting tool to form a desired window cut 60 in an outer sleeve 50.

While the slit cut guide 72 shown in FIGS. 25-28 includes a second cylindrical body 80 with six V-shaped grooves 82 circumferentially disposed about the second cylindrical body 80, it is contemplated that the slit cut guide 72 can include four V-shaped grooves, eight V-shaped grooves, or any number of V-shaped grooves suitable to guide a cutting tool to form a desired number of window cuts 60 in the outer sleeve 50.

The example shown in FIGS. 25-28 shows the slit cut guide 72 coupled to the first end 86 of the central body 84 and the window cut guide 74 coupled to the second side of the central body 84. In other implementations, the slit cut guide 72 and the window cut guide 74 are coupled directly to each other with no central body or formed as separate tools that are not coupled together.

The sheath 8 disclosed herein can be made using the following methods. The inner sleeve 40 is provided, which has the proximal end 42, the distal end 44, and the inner sleeve body 46 that extends between the proximal end 42 and the distal end 44 defining a central opening 48 as described above. The outer sleeve 50 is also provided, which has the proximal end 52, the distal end 54 and the outer sleeve body 56 that extends between the proximal end 52 and the distal end 54 defining a central opening 58 as described above. The inner sleeve 40 and the outer sleeve 50 are provided in an un-flared and uncut configuration as shown in FIGS. 33-34 (dashed lines 49 and 59 showing the representative locations of the cut lines for slits 49, 59). If desired, the outer sleeve 50 can be formed including the recesses 62 as shown in FIGS. 34-36, e.g., by extrusion. In another example, the recesses 62 are added or cut into the outer sleeve 50 after it is formed, e.g., cutting, laser etching.

The longitudinal slits 49, 59 and window cuts 60 are then formed in the inner sleeve 40 and the outer sleeve 50. A cutting guide 70 (FIGS. 25-28) is used to cut the longitudinal slits 49 into the proximal end 42 of the inner sleeve 40 and the longitudinal slits 59 into the proximal end 52 of the outer sleeve 50 (e.g., three longitudinal slits 49 and three longitudinal slits 59). The cutting guide 70 is also used to cut the window cuts 60 into the distal end 54 of the outer sleeve 50 (e.g., six window cuts 60).

During cutting, the proximal end 42 of the inner sleeve 40 is advanced over the slit cut guide 72 of the cutting guide 70 such that the slit cut guide 72 is disposed inside the central opening 48 of the inner sleeve 40. A cutting tool (e.g., blade) is advanced through the inner sleeve 40 into each of the plurality of grooves 78 at the proximal end 42 of the inner sleeve 40. The cutting tool is advanced along the axial length of each longitudinal grooves 78, cutting the plurality of longitudinal slits 49 into the inner sleeve body 46. Each of the plurality of longitudinal slits 49 of the inner sleeve 40 extend along a line generally parallel with a longitudinal axis of the inner sleeve 40.

The proximal end 52 of the outer sleeve 50 is advanced over the slit cut guide 72 of the cutting guide 70 such that the slit cut guide 72 is disposed inside the central opening 58 of the outer sleeve 50. The cutting tool is advanced through the outer sleeve 50 into each of the plurality of grooves 78 at the proximal end 52 of the outer sleeve 50. The cutting tool is advanced along the axial length of each longitudinal groove 78, cutting the plurality of longitudinal slits 59 into the outer sleeve body 56. Each of the plurality of longitudinal slits 59 of the outer sleeve 50 extend along a line generally parallel with a longitudinal axis of the outer sleeve 50.

The distal end 54 of the outer sleeve 50 is advanced over the window cut guide 74 of the cutting guide 70 such that the window cut guide 74 is disposed inside the central opening 58 of the outer sleeve 50. The cutting tool is advanced through the outer sleeve 50 into each of the plurality of V-shaped grooves 82 at the distal end of the window cut guide 74. The cutting tool is advanced along the V-shaped edges of each V-shaped groove 82, cutting the plurality of window cuts 60 into the outer sleeve body 56. If desired, the cutting tool is advanced along the planar groove segment to form the distal end of the segment 51 described above. Each of the slits 59 is cut to a length that is greater than the length of each window cut 60, such that placing the inner sleeve 40 and the outer sleeve 50 over the flaring tool 90 causes the slits 59 to expand and create the tapered shape of the coupled proximal end 32.

The inner sleeve 40 and the outer sleeve 50 are then secured together. To secure the outer sleeve 50 to the inner sleeve 40, the outer sleeve 50 is disposed over the proximal end 42 of the inner sleeve 40 such that the proximal ends 42, 52 of the inner sleeve 40 and the outer sleeve 50 are axially aligned. The outer sleeve 50 is rotationally disposed on the inner sleeve 40 such that each of the longitudinal slits 59 of the outer sleeve 50 are circumferentially aligned with a solid portion of the inner sleeve 40 that does not include a longitudinal slit 49. As such the slits 49 of the inner sleeve 40 are circumferentially spaced from the slits 59 of the outer sleeve 50.

In some implementations the outer sleeve 50 is coupled to the inner sleeve 40 by tacking the outer sleeve 50 to the inner sleeve 40. This secures the outer sleeve 50 to the inner sleeve 40 in a desired longitudinal and circumferential orientation prior to heat processing, e.g., reflowing. The tacking can be done by a soldering gun or any other mechanism for locally melting/reflowing precise areas of inner sleeve 40 and the outer sleeve 50 together.

In some implementations the outer sleeve 50 and the inner sleeve 40 can also be held in a desired position by disposing a heat shrink sleeve around the inner sleeve 40 and the outer sleeve 50 at a location that includes the proximal end 42 of the inner sleeve 40. The heat shrink sleeve compresses the inner sleeve 40 and the outer sleeve 50 in a desired orientation and is removed after reflowing of the sheath 8. The heat shrink sleeve is disposed around the coupled proximal end 32 when reflowing the inner sleeve 40 and the outer sleeve 50. The heat shrink sleeve promotes even circumferential and longitudinal material distribution for a consistent material thickness. Example heat shrink material includes Fluorinated Ethylene Propylene (FEP) heat shrink or any other heat shrink capable of compressing an sheath.

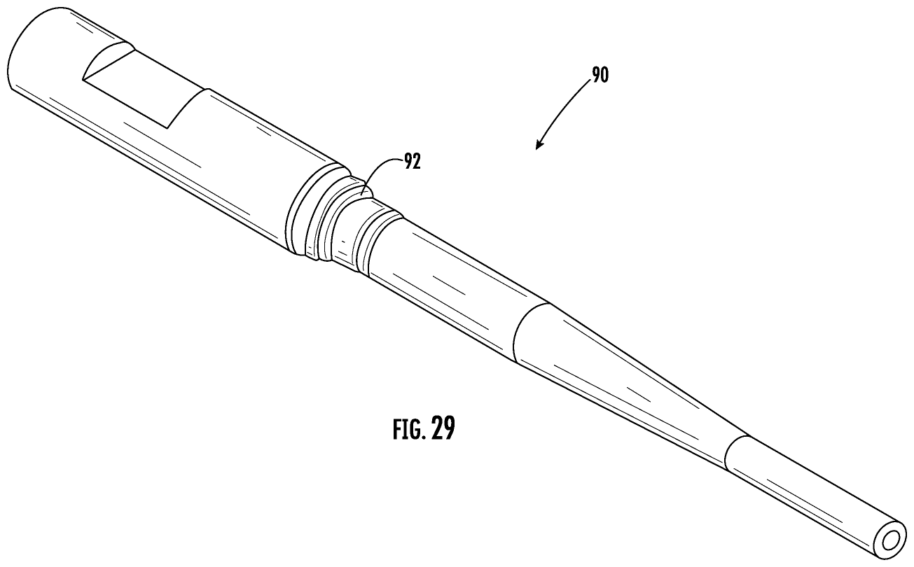

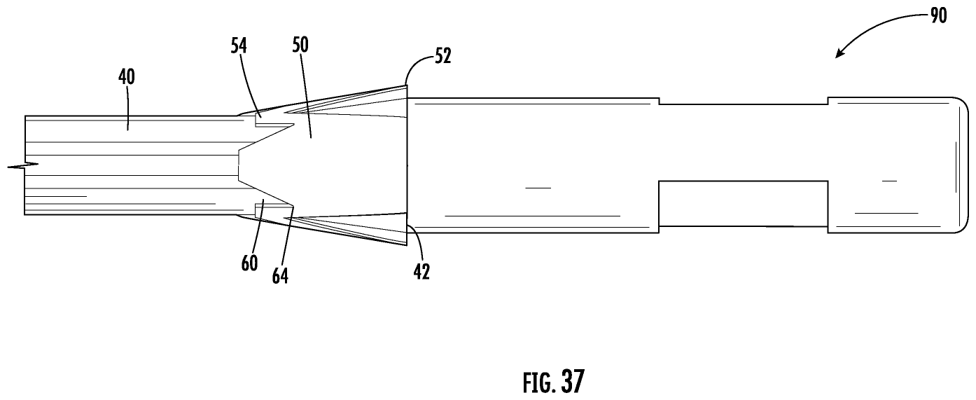

The proximal end 32 of the sheath 8 is then flared to an expanded configuration. As illustrated in FIG. 37, the proximal end 42 of the inner sleeve 40 is disposed over the flaring tool 90 (FIGS. 29-32), such that a portion of the flaring tool 90 is disposed inside a portion of the central opening 48 of the inner sleeve 40. The flaring tool 90 has a tiered structure of increasing diameters 92, such that the diameter of the flaring tool 90 increases in the proximal direction. As shown in FIG. 37, the proximal ends 42, 52 of the inner sleeve 40 and the outer sleeve 50 are advanced in the proximal direction over a portion of the flaring tool 90. As the proximal ends 42, 52 of the inner sleeve 40 and outer sleeve 50 are advanced over the flaring tool 90, the proximal end 42 of the inner sleeve 40 and the proximal end 52 of the outer sleeve 50 are radially expanded.

The outer sleeve 50 and the inner sleeve 40 coupled together forming a coupled proximal end 32. The outer sleeve 50 and the inner sleeve 40 (mounted on the flaring tool 90 or other support mandrel) are placed in an oven and reflowed together. For example, the combined outer sleeve 50 and inner sleeve 40 are heated in an oven maintained at a temperature ranging from 400 degrees Fahrenheit to 500 degrees Fahrenheit. In some examples, the oven is maintained at a temperature of 450 degrees Fahrenheit. The combined outer sleeve 50 and inner sleeve 40 are heated for a period of time ranging from 1 minute to 2.5 minutes. For example, the combined outer sleeve 50 and inner sleeve 40 are heated for 1.5 minutes. In some examples, the combined outer sleeve 50 and inner sleeve 40 are heated are heated for 1.5 minutes in an oven maintained at a temperature of 450 degrees Fahrenheit. The reflowed outer sleeve 50 and inner sleeve 40 form a uniform thickness about the circumference of the coupled proximal end 32. The reflow process melts/fills the slits 49 of the inner sleeve 40 with portions of the outer sleeve body 56 and melts/fills the slits 59 and window cuts 60 of the outer sleeve 50 with portions of the inner sleeve body 46 forming a uniform circumference at the coupled proximal end 32. During reflow the coupled proximal end 32 becomes malleable and conforms to the diameters of the flaring tool 90 which increases in the proximal direction. As the coupled proximal end 32 is reflowed, the proximal end 32 forms a fillet/rounded surface transition that has a smooth outer surface on the coupled proximal end 32. As such, reflowing of the coupled proximal end 32 around the flaring tool 90 forms the flared diameter at the coupled proximal end 32. Although the coupled proximal end 32 is formed using a reflow oven, in other implementations, the coupled proximal end 32 is formed using soldering, or any other method of bonding the sheath layers together.

As described above, the sheath 8 can then be coupled to the sheath hub 20. As illustrated in FIGS. 8-10, the coupled proximal end 32 is advanced over the distal end of the sheath hub 20 such that the sheath hub 20 is disposed within the diameter of the flared proximal end 32 of the sheath 8. The sheath hub cap 22 is advanced proximally along the length of the sheath 8. The sheath hub cap 22 is screwed onto the sheath hub 20 to secure the sheath hub cap 22 to the sheath hub 20. Screwing the sheath hub cap 22 to the sheath hub 20 sandwiches the coupled proximal end 32 between an inner diameter of the sheath hub cap 22 and an outer diameter of the sheath hub 20 as shown in FIG. 10. As such, the sheath 8 is secured to the sheath hub 20 by an interference fit. Although the sheath hub 20 as shown in FIG. 10 is secured using an interference fit, the sheath 8 can be secured to the sheath hub 20 by applying adhesive or any other method of securing the sheath 8 and sheath hub 20 together.

Exemplary Aspects:

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An dilator for use with a medical device delivery system, the dilator comprising: a shaft with a tapered distal region, the tapered distal region narrowing distally towards a distal tip; an expander positioned on the shaft adjacent to the distal tip, the expander comprising a compressed configuration wherein the expander is compressed against the shaft and an expanded configuration wherein the expander extends radially away from the shaft; and a proximal region comprising a stopping feature, the stopping feature positioned a fixed distance from the expander.

Example 2: The dilator according to any example herein, particularly example 1, wherein the expander is coupled to the shaft.

Example 3: The dilator according to any example herein, particularly examples 1-2, wherein the expander is positioned proximal to the tapered distal region.

Example 4: The dilator according to any example herein, particularly examples 1-3, wherein the expander comprises at least one tubular portion that extends circumferentially around the shaft.

Example 5: The dilator according to any example herein, particularly example 4, wherein the at least one tubular portion of the expander is bonded to the shaft.

Example 6: The dilator according to any example herein, particularly examples 1-5, wherein the expander comprises a shape memory material and is configured to take the expanded configuration upon a change in the surroundings.

Example 7: The dilator according to any example herein, particularly examples 1-6, wherein the expander comprises a plurality of longitudinally extending slits.

Example 8: The dilator according to any example herein, particularly example 7, wherein the plurality of longitudinally extending slits extends proximally from a distal end of the expander.

Example 9: The dilator according to any example herein, particularly examples 7 or 8, wherein the plurality of longitudinally extending slits divides the expander into longitudinally extending arms that move away from the shaft upon a change in the surroundings.

Example 10: The dilator according to any example herein, particularly example 1-6, wherein the expander comprises one or more folds in the compressed configuration that unfold upon a change in the surroundings.

Example 11: The dilator according to any example herein, particularly example 10, wherein the expander forms a funnel in the expanded configuration.

Example 12: The dilator according to any example herein, particularly examples 1-6, wherein the expander comprises two or more extensions pointing toward the proximal region of the dilator.

Example 13: The dilator according to any example herein, particularly example 12, wherein the expander is biased toward the expanded configuration, and wherein the extensions extend radially away from the shaft.

Example 14: The dilator according to any example herein, particularly examples 12 or 13, wherein each of the two or more extensions comprises a hinge coupling the extension to the expander.

Example 15: The dilator according to any example herein, particularly example 14, wherein each of the two or more extensions is rotatable around the hinge to point toward the distal region of the dilator.

Example 16: The dilator according to any example herein, particularly examples 1-15, wherein in the expanded configuration, the expander extends away from the shaft in multiple radial directions.

Example 17: The dilator according to any example herein, particularly examples 1-16, wherein in the expanded configuration, the expander extends away from the shaft in all radial directions.

Example 18: The dilator according to any example herein, particularly examples 1-17, wherein the stopping feature has a larger diameter than the shaft.

Example 19: The dilator according to any example herein, particularly examples 1-18, wherein the stopping feature is a visual indicator.

Example 20: A medical device delivery system comprising: an dilator, the dilator comprising: a shaft with a tapered distal region, the tapered distal region narrowing distally towards a distal tip; an expander positioned on the shaft adjacent to the distal tip, the expander comprising a compressed configuration wherein the expander is compressed against the shaft and an expanded configuration wherein the expander is extending radially away from the shaft; and a proximal region comprising a stopping feature, the stopping feature positioned a fixed length from the expander; and a sheath comprising a distal portion, the distal portion configured to expand radially outward.

Example 21: The medical device delivery system according to any example herein, particularly example 20, wherein the expander comprises two or more extensions pointing toward the proximal region of the dilator.

Example 22: The medical device delivery system according to any example herein, particularly example 21, wherein the expander is biased toward the expanded configuration, and wherein the extensions extend radially away from the shaft.

Example 23: The medical device delivery system according to any example herein, particularly examples 21 or 22, wherein each of the two or more extensions comprises a hinge coupling the extension to the expander.

Example 24: The medical device delivery system according to any example herein, particularly example 23, wherein each of the two or more extensions is rotatable around the hinge to point toward the distal region of the dilator.

Example 25: The medical device delivery system according to any example herein, particularly examples 20-24, wherein the sheath comprises two or more weakened longitudinal areas.

Example 26: The medical device delivery system according to any example herein, particularly example 25, wherein the sheath comprises a proximal portion having a first alignment indicator and the dilator comprises a proximal region having a second alignment indicator, and wherein alignment of the first alignment indicator with the second alignment indicator corresponds to alignment of the weakened areas of the distal portion of the sheath with the extensions of the expander.

Example 27: A method of making a dilator, the method comprising: forming a shaft with a tapered distal region; coupling an expander to the shaft adjacent to a distal tip of the shaft; and adding a stopping feature to a proximal region of the shaft.

Example 28: The method of making a dilator according to any example herein, particularly example

Example 29: The medical device delivery system according to any example herein, particularly example 27 or 28, wherein coupling the expander to the shaft comprises coupling a tubular portion of the expander such that it extends circumferentially around the shaft.

Example 30: The method of making a dilator according to any example herein, particularly examples 27-29, further comprising forming the expander of a shape memory material.

Example 31: The method of making a dilator according to any example herein, particularly examples 27-30, further comprising cutting longitudinally extending slits extending proximally from a distal end of the expander.

Example 32: The method of making a dilator according to any example herein, particularly examples 27-30, further comprising forming a funnel with the shape memory material on a distal side of the expander.

Example 33: The method of making a dilator according to any example herein, particularly example 32, further comprising folding the funnel to a tubular shape.

Example 34: The method of making a dilator according to any example herein, particularly examples 27-30, further comprising forming the expander to include two or more extensions biased to point in a proximal direction and extend radially away from the shaft, wherein the extensions are rotatable about a hinge to point in a distal direction.

Example 35: The method of making a dilator according to any example herein, particularly examples 27-34, further comprising forming the stopping feature to have a larger diameter than the shaft.

Example 36: The method of making a dilator according to any example herein, particularly examples 27-35, wherein adding a stopping feature comprises marking a stopping point as a visual indicator on the shaft.

Example 37: A method of delivering a medical device to a subject, the method comprising: inserting a tapered dilator into a sheath, the tapered dilator comprising an expander; inserting the tapered dilator and the sheath into the vasculature of the subject; expanding or tearing a distal portion of the sheath with the expander; removing the dilator from the sheath; and delivering the medical device to the subject through the sheath.

Example 38: The method of delivering a medical device according to any example herein, particularly example 37, wherein inserting the tapered dilator into the sheath comprises axially translating the dilator until a stopping feature on the dilator is a predetermined distance from a distal end of the sheath.

Example 39: The method of delivering a medical device according to any example herein, particularly examples 37 or 38, wherein inserting the tapered dilator into the sheath comprises axially translating the dilator until the stopping feature abuts a proximal end of the sheath.

Example 40: The method of delivering a medical device according to any example herein, particularly examples 37-39, wherein inserting the tapered dilator into the sheath comprises axially translating the dilator until at least part of the expander is beneath the distal portion of the sheath.

Example 41: The method delivering a medical device according to any example herein, particularly examples 37-40, wherein expanding the distal portion of the sheath comprises inducing the expander to take an expanded configuration beneath the distal portion of the sheath.

Example 42: The method delivering a medical device according to any example herein, particularly examples 37-41, wherein inducing the expander to take an expanded configuration comprises moving longitudinally extending arms of the expander radially away from a shaft of the dilator.

Example 43: The method delivering a medical device according to any example herein, particularly examples 37-41, wherein inducing the expander to take an expanded configuration comprises unfolding one or more folds of the expander.

Example 44: The method delivering a medical device according to any example herein, particularly examples 37-43, wherein inserting the tapered dilator into the sheath comprises axially translating the dilator until at least part of the expander is distal to the distal portion of the sheath.

Example 45: The method delivering a medical device according to any example herein, particularly example 44, wherein removing the dilator from the sheath further comprises tearing the distal portion of the sheath with the expander.

Example 46: The method delivering a medical device according to any example herein, particularly example 45, further comprising aligning the expander before tearing the distal portion and targeting the tearing to weakened areas of the distal portion of the sheath.

Example 47: The method delivering a medical device according to any example herein, particularly examples 44-46, wherein the part of the expander distal to the distal portion of the sheath is two or more extensions biased toward a radially expanded configuration wherein the extensions point proximally, and wherein removing the dilator from the sheath comprises rotating the two or more extensions to point distally and compressing the expander as it is proximally retracted through the sheath.

Example 48: A method of making a sheath, the method comprising: forming an inner sleeve having a proximal end, a distal end, and an inner sleeve body that extends between the proximal end and the distal end defining a central opening; forming an outer sleeve having a proximal end, a tapered distal end and an outer sleeve body that extends between the proximal end and the distal end defining a central opening; forming a plurality of longitudinal slits in the inner sleeve, the slits extending from the proximal end of the inner sleeve along a portion of the inner sleeve body; forming a plurality of longitudinal slits in the outer sleeve, the slits extending from the proximal end of the outer sleeve along a portion of the outer sleeve body; forming a plurality of window cuts in the outer sleeve, the window cuts extending from the distal end of the outer sleeve along a portion of the outer sleeve body; disposing the proximal end of the inner sleeve over a flaring tool; disposing a portion of the inner sleeve in the central opening of the outer sleeve; and coupling the outer sleeve and the inner sleeve together forming a coupled proximal end, wherein coupling the outer sleeve and the inner sleeve together forms a uniform thickness about the circumference of the coupled proximal end.

Example 49: The method of making a sheath according to any example herein, particularly example 48, wherein the outer sleeve is coupled to the inner sleeve by heat processing.

Example 50: The method of making a sheath according to any example herein, particularly example 48 or 49, wherein the outer sleeve reflowed with the inner sleeve.

Example 51: The method of making a sheath according to any example herein, particularly examples 48-50, wherein the outer sleeve and the inner sleeve are reflowed around the flaring tool forming a flared surface at the coupled proximal end.

Example 52: The method of making a sheath according to any example herein, particularly examples 48-51, further comprising forming a fillet surface transition at the coupled proximal end.

Example 53: The method of making a sheath according to any example herein, particularly examples 48-52, further comprising longitudinally aligning the proximal end of the inner sleeve and the proximal end of the outer sleeve.

Example 54: The method of making a sheath according to any example herein, particularly examples 48-53, further comprising positioning the outer sleeve such that the slits of the inner sleeve are circumferentially spaced from the slits of the outer sleeve.

Example 55: The method of making a sheath according to any example herein, particularly examples 48-54, wherein forming a plurality of longitudinal slits in the inner sleeve comprises placing the proximal end of the inner sleeve over a split cut guide and sliding a cutting tool along the slit cut guide.

Example 56: The method of making a sheath according to any example herein, particularly examples 48-55, wherein each of the plurality of longitudinal slits of the inner sleeve extend along a line generally parallel with a longitudinal axis of the inner sleeve.

Example 57: The method of making a sheath according to any example herein, particularly examples 48-56, wherein forming a plurality of longitudinal slits in the inner sleeve comprises placing the proximal end of the inner sleeve over a slit cut guide and sliding a cutting tool along the slit cut guide.

Example 58: The method of making a sheath according to any example herein, particularly examples 48-57, wherein forming a plurality of longitudinal slits in the outer sleeve comprises placing the proximal end of the outer sleeve over a slit cut guide and sliding a cutting tool along the slit cut guide.

Example 59: The method of making a sheath according to any example herein, particularly examples 48-58, wherein each of the plurality of longitudinal slits of the outer sleeve extend along a line generally parallel with a longitudinal axis of the outer sleeve.

Example 60: The method of making a sheath according to any example herein, particularly examples 48-59, wherein forming a plurality of longitudinal slits in the outer sleeve comprises placing the proximal end of the outer sleeve over a slit cut guide and sliding a cutting tool along the slit cut guide.

Example 61: The method of making a sheath according to any example herein, particularly examples 48-60, wherein the length of each of the slits is greater than the length of each window cut, such that placing the inner sleeve and the outer sleeve over the flaring tool causes the slits to expand and create the tapered shape of the coupled proximal end.

Example 62: The method of making a sheath according to any example herein, particularly examples 48-61, wherein forming a plurality of window cuts in the outer sleeve comprises placing the distal end of the outer sleeve over a window cut guide and sliding a cutting tool along the window cut guide.

Example 63: The method of making a sheath according to any example herein, particularly examples 48-62, further comprising tacking the outer sleeve to the inner sleeve.

Example 64: The method of making a sheath according to any example herein, particularly examples 48-63, further comprising disposing a heat shrink sleeve around the outer sleeve and a portion of the inner sleeve that includes the proximal end of the inner sleeve.

Example 65: The method of making a sheath according to any example herein, particularly example 64, wherein the heat shrink is PTFE heat shrink.

Example 66: The method of making a sheath according to any example herein, particularly examples 64 or 65, further comprising removing the heat shrink sleeve.

Example 67: The method of making a sheath according to any example herein, particularly examples 48-66, further comprising disposing the coupled proximal end between a sheath hub and a sheath hub cap.

Example 68: The method of making a sheath according to any example herein, particularly example 67, wherein disposing the coupled proximal end between the sheath hub and the sheath hub cap secures the proximal end to the sheath hub.

Example 69: A sheath comprising: an inner sleeve having a flared proximal end, a distal end and an inner sleeve body that extends between the proximal end and the distal end and defines a central opening, the inner sleeve comprising a plurality of circumferentially spaced slits extending from the proximal end along a portion of the inner sleeve body; and an outer sleeve having a proximal end, a tapered distal end and an outer sleeve body that extends between the proximal end and the distal end and defines a central opening, the outer sleeve comprising a plurality of circumferentially spaced slits extending from the proximal end along a portion of the outer sleeve body, and further comprising a plurality of window cuts that extend from the distal end along a portion of the outer sleeve body, wherein a portion of the proximal end of the inner sleeve is disposed in the central opening of the outer sleeve such that the proximal end of the inner sleeve and the proximal end of the outer sleeve are longitudinally aligned, and wherein the inner sleeve and the outer sleeve are coupled together forming a coupled proximal end.

Example 70: The sheath according to any example herein, particularly example 69, wherein the outer sleeve further comprises a recess along a length of outer sleeve that extends circumferentially from either side of each of the circumferentially spaced slits.

Example 71: The sheath according to any example herein, particularly example 69 or 70, wherein the window cuts are circumferentially spaced apart from the slits around the outer sleeve.

Example 72: The sheath according to any example herein, particularly examples 69-71, wherein a window cut is disposed on either side of each slit.

Example 73: The sheath according to any example herein, particularly examples 69-72, wherein each slit is centered between an adjacent pair of window cuts.

Example 74: The sheath according to any example herein, particularly examples 69-73, wherein a length of each slit is greater than a length of each window cut.

Example 75: The sheath according to any example herein, particularly examples 69-74, wherein each slit extends along the outer sleeve body beyond a proximal end of each cut.

Example 76: The sheath according to any example herein, particularly examples 69-75, wherein each of the plurality of longitudinal slits of the inner sleeve extend along a line generally parallel with a longitudinal axis of the inner sleeve.

Example 77: The sheath according to any example herein, particularly examples 69-76, wherein each of the plurality of longitudinal slits of the inner sleeve has a same length along the inner sleeve.

Example 78: The sheath according to any example herein, particularly examples 69-77, wherein each of the plurality of longitudinal slits of the outer sleeve extend along a line generally parallel with a longitudinal axis of the outer sleeve.

Example 79: The sheath according to any example herein, particularly examples 69-78, wherein each of the plurality of longitudinal slits of the outer sleeve has a same length along the outer sleeve.

Example 80: The sheath according to any example herein, particularly examples 69-79, wherein the slits of the inner sleeve are circumferentially spaced apart from the slits in the outer sleeve.

Example 81: The sheath according to any example herein, particularly examples 69-80, wherein the number of slits in the inner sleeve is equal to the number of slits in the outer sleeve.

Example 82: The sheath according to any example herein, particularly examples 69-81, wherein the number of slits in the outer sleeve is equal to the number of window cuts in the outer sleeve.

Example 83: The sheath according to any example herein, particularly examples 69-82, wherein there are twice as many longitudinal slits as window cuts on the outer sleeve.

Example 84: The sheath according to any example herein, particularly examples 69-83, wherein the plurality of slits in the inner sleeve comprises 3 slits.

Example 85: The sheath according to any example herein, particularly examples 69-83, wherein the plurality of slits in the inner sleeve comprises 4 slits.

Example 86: The sheath according to any example herein, particularly examples 69-85, wherein the plurality of slits in the outer sleeve comprises 3 slits.

Example 87: The sheath according to any example herein, particularly examples 69-85, wherein the plurality of slits in the outer sleeve comprises 4 slits.

Example 88: The sheath according to any example herein, particularly examples 69-87, wherein the plurality of window cuts in the outer sleeve comprises 3 window cuts.

Example 89: The sheath according to any example herein, particularly examples 69-87, wherein the plurality of window cuts in the outer sleeve comprises 4 window cuts.

Example 90: The sheath according to any example herein, particularly examples 69-89, wherein the coupled proximal end comprises a flared diameter.

Example 91: The sheath according to any example herein, particularly examples 69-90, wherein the flared diameter further comprises a fillet transition surface.

Example 92: The sheath according to any example herein, particularly examples 69-91, wherein the inner sleeve and the outer sleeve each have a uniform thickness.

Example 93: The sheath according to any example herein, particularly examples 69-92, wherein the uniform thickness is double the thickness of the inner sleeve.

Example 94: The sheath according to any example herein, particularly examples 69-93, wherein the inner sleeve and outer sleeve are formed from the same material.

Example 95: The sheath according to any example herein, particularly examples 69-94, further comprising a sheath hub and a sheath hub cap coupled to the coupled proximal end.

Example 96: A cutting guide device, the device comprising: a slit cut guide comprising a first cylindrical body and a plurality of longitudinal grooves circumferentially disposed about the first cylindrical body; a window cut guide comprising a second cylindrical body and plurality of V-shaped grooves circumferentially disposed about the second cylindrical body; a central body, wherein the slit cut guide and the window cut guide are each coupled to the central body, wherein each of the plurality of longitudinal grooves define a guide channel shaped to guide a cutting tool to form a longitudinal slit in a tube disposed about the slit cut guide, and wherein each of the plurality of V-shaped grooves defines guide edges to guide a cutting tool to form a window cut in a tube disposed about the window cut guide.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims. can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A medical device delivery system comprising a dilator, the dilator comprising:
a shaft with a tapered distal region, the tapered distal region narrowing distally towards a distal tip;
an expander positioned on the shaft adjacent to the distal tip, the expander comprising a compressed configuration wherein the expander is compressed against the shaft and an expanded configuration wherein the expander extends radially away from the shaft; and
a proximal region comprising a stopping feature, the stopping feature positioned a fixed distance from the expander.

2. The medical device delivery system of claim 1, wherein the expander is coupled to the shaft.

3. The medical device delivery system of claim 1, wherein the expander is positioned proximal to the tapered distal region.

4. The medical device delivery system of claim 1, wherein the expander comprises at least one tubular portion that extends circumferentially around the shaft.

5. The medical device delivery system of claim 1, wherein the expander comprises a shape memory material and is configured to take the expanded configuration upon a change in the surroundings.

6. The medical device delivery system of claim 1, wherein the expander comprises a plurality of longitudinally extending slits.

7. The medical device delivery system of claim 6, wherein the plurality of longitudinally extending slits extends proximally from a distal end of the expander.

8. The medical device delivery system of claim 6, wherein the plurality of longitudinally extending slits divides the expander into longitudinally extending arms that move away from the shaft upon a change in a set of surroundings.

9. The medical device delivery system of claim 1, wherein the expander comprises one or more folds in the compressed configuration that unfold upon a change in a set of surroundings.

10. The medical device delivery system of claim 9, wherein the expander forms a funnel in the expanded configuration.

11. The medical device delivery system of claim 1, wherein the expander comprises two or more extensions pointing toward the proximal region of the dilator.

12. The medical device delivery system of claim 11, wherein the expander is biased toward the expanded configuration, and wherein the two or more extensions extend radially away from the shaft.

13. The medical device delivery system of claim 11, wherein each of the two or more extensions is rotatable around a hinge to point toward the tapered distal region of the dilator.

14. The medical device delivery system of claim 1, wherein in the expanded configuration, the expander extends away from the shaft in multiple radial directions.

15. The medical device delivery system of claim 1, wherein the stopping feature has a larger diameter than the shaft.

16. The medical device delivery system of claim 1, wherein the stopping feature is a visual indicator.

17. The medical device delivery system of claim 1, further comprising a sheath comprising a distal portion, the distal portion configured to expand radially outward.

18. The medical device delivery system of claim 17, wherein the sheath comprises two or more weakened longitudinal areas.

19. The medical device delivery system of claim 18, wherein the sheath comprises a proximal portion having a first alignment indicator and the dilator comprises a proximal region having a second alignment indicator, and wherein alignment of the first alignment indicator with the second alignment indicator corresponds to alignment of the weakened areas of the distal portion of the sheath with the two or more extensions of the expander.

20. A method of delivering a medical device to a subject, the method comprising:
inserting a tapered dilator into a sheath, the tapered dilator comprising an expander positioned adjacent a distal tip of the dilator and a stopping feature positioned a fixed distance from the expander;
inserting the tapered dilator and the sheath into a vasculature of the subject;
expanding or tearing a distal portion of the sheath with the expander;
removing the dilator from the sheath; and
delivering the medical device to the subject through the sheath.
